(19) United States
(12) Patent Application Publication
(10) Pub. No.: US 2003/0056234 A1
(43) Pub. Date: Mar. 20, 2003

(54) Polymorphic sequences of the human ABCA1 gene, their uses, and detection methods and kits therefor

(76) Inventors: Patrice Denefle, Saint Maur (FR); Marie-Francoise Rosier-Montus, Antony (FR); Isabelle Arnould-Reguigne, Chennevieres Sur Marne (FR); Nicolas Duverger, Paris (FR); Francois Cambien, Paris (FR)

Correspondence Address: FINNEGAN, HENDERSON, FARABOW, GARRETT &, DUNNER LLP, 1300 I STREET, NW, WASHINGTON, DC, 20005, US

(21) Appl. No.: 09/984,827

(22) Filed: Oct. 31, 2001

Related U.S. Application Data
(60) Provisional application No. 60/254,108, filed on Dec. 11, 2000.

(30) Foreign Application Priority Data
Oct. 31, 2000 (FR) 00/14037

(51) Int. Cl. A01K 67/00; C07H 21/04; C12P 21/02; C12N 5/06; C07K 14/435

(52) U.S. Cl. 800/8; 536/23.5; 530/350; 435/69.1; 435/325; 435/320.1

(57) ABSTRACT

The present invention relates to isolated nucleic acids encoding the ABCA1 transporter protein and comprising polymorphic sequence variations, and to peptides which are derived from the human ABCA1 transporter and which contain polymorphic amino acids.

The invention also relates to allele-specific primers and probes which hybridize to regions flanking or containing these polymorphic positions or sites, to methods and kits or packs for analyzing the differences in allelism which affect the ABCA1 gene, and finally to the use of the polymorphisms of the human ABCA1 gene for diagnosing a disease, or a predisposition to a disease, in particular linked to the plasma HDL (High Density Lipoprotein) cholesterol concentration, as is, for example, the case of familial HDL deficiencies such as Tangier disease, myocardial infarction, atherosclerosis and other cardiovascular disorders.

The present invention relates to polymorphisms of the human ABCA1 gene and the novel polypeptide alleles encoded by the various sequences exhibiting the allelic variations. The invention also relates to methods and kits intended for analyzing the allelic variations in the human ABCA1 gene, and to the use of the ABCA1 polymorphism for diagnosing and treating cardiovascular disorders such as myocardial infarction, atherosclerosis or Tangier disease.

ABCA1 is a member of the superfamily of ABC (ATP Binding Cassette) transporter proteins which are involved in the transport of peptides, of sugars, of vitamins or of steroid hormones (Dean et al. Curr. Opin. Genet. 5 (1995) 779-785; Decocottignies et al. Nat. Genet. Dev., 15 (1997) 137-145; Allikmets et al., Hum. Mol. Genet, 5 (1996) 1649-1655).

The members of this transporter family which are, firstly, extremely conserved through evolution, from bacteria to humans, have, secondly, a common general structure characterized by two nucleotide binding folds (or NBFs) with Walker A and B motifs and two transmembrane domains, each of the transmembrane domains consisting of several helixes. The specificity of the ABC transporters for the various molecules transported appears to be determined by the structure of the transmembrane domains, while the energy required for the transport activity is provided by the degradation of the ATP in the NBF fold.

The transport of cholesterol from the liver to the tissues, and vice versa, is carried out by the two lipoprotein complexes, namely LDLs (Low Density Lipoproteins) and HDLs (High Density Lipoproteins). LDLs supply the tissues with cholesterol whereas, on the other hand, HDLs take the excess cholesterol formed in the tissues to the liver. During transport, the cholesterol is acylated by transferring lecithin fatty acids via the action of the enzyme phosphatidyl sterol acyltransferase (LCAT).

It is now clearly established in patients with low plasma HDL levels that high LDL levels lead to an accumulation of cholesterol in the vessels and the probability that coronary insufficiency will appear (Castelli et al., Jama (1986) 256(20), 2835-8). Specifically, slower elimination of excess cholesterol in the tissues promotes the formation of arterial plaques, and in the end increases the risk of heart attack, of angina pectoris or of a disorder of the peripheral arterial system.

These lipoprotein complexes, LDLs and HDLs, which are involved in, respectively, the mechanisms of inflow and outflow of cholesterol, from the liver to the tissues or vice versa, have a spherical structure of different density which is composed essentially of a core of apolar lipids consisting of triacylglycerols and of cholesterol esters and of a crown consisting of apolipoproteins and of amphiphilic lipids.

It is generally accepted that the outflow of cholesterol from the tissues toward the liver is carried out via two different pathways. A first pathway, termed “passive”, promotes the outflow of cholesterol from the plasma membrane to the HDLs, while the second pathway is energy-dependent and uses in particular the apolipoprotein A-I (Apo-AI) of the nascent HDLs, which is probably recognized and binds to cellular membrane-bound receptors so as to allow the translocation of the excess cholesterol to the HDL particles (Rothblat et al., J. Lipid. Res. (1999) 40(5) 781-96). It has recently been demonstrated in particular that fibroblasts and macrophages are the center of active translocation of cholesterol using lipid-depleted apolipoproteins, such as ApoA-I, ApoA-II and Apo-E (Yokoyama S., Biochim. Biophys. Acta (1998) 1392(1), 1-15; Takahashi et al., PNAS (1999) 96(20), 11358-63).

Various diseases linked to an HDL deficiency, and in particular a deficiency of the active pathway of cholesterol translocation, have been described. These diseases include Tangier disease and familial HDL deficiency (FHD) (Remaley et al., Arterioscl. Thromb. Vasc. Biol (1997) 17(9) 1813-21; Marcil et al., Arterioscl. Thromb. Vasc. Biol. (1999) 19(1), 159-69; Francis et al., (1995) J. Clin. Invest. 96(1), 78-87).

Tangier disease appears to be linked to a cellular deficiency in the active pathway of cellular cholesterol translocation, which leads to degradation of HDLs and to a disturbance of lipoprotein metabolism. Specifically, particles which do not incorporate cholesterol from the peripheral cells and which cannot be correctly metabolized are rapidly removed from the body. The plasma concentration of circulating HDL of these patients is extremely low and the HDLs no longer, therefore, ensure that the cholesterol is returned to the liver. The excess cholesterol accumulated in the peripheral cells and tissues causes characteristic clinical signs such as the formation of orange-colored tonsils (Serfaty-Lacrosniere et al., (1994) Athérosclerosis 107(1) 85-98).

The familial disorders linked to HDL metabolism are also characterized by a low concentration of HDL particles, and are most commonly detected in patients suffering from coronary diseases (Marcil et al., The Lancet (1999) 354(9187), 1341-6). This cardioprotective effect of HDL is probably explained by its involvement in the transport of cholesterol from the peripheral tissues toward the liver (Bruce et al., Annu. Rev. Nutr. (1998) 18, 297-3130).

The product of the ABCA1 gene plays a role in the regulation of the cellular metabolism of cholesterol and in the reverse transport of cholesterol and of phospholipids. In this respect, it has recently been demonstrated that the ABCA1 gene is a key gene in the active pathway of cholesterol translocation from cells to HDLs. Specifically, it has been observed that the ABCA1 transporter is mutated in patients in which the reverse transport of cholesterol is affected and, in particular, in patients suffering both from Tangier disease and from familial HDL deficiency (Lawn et al., J. Clin. Invest. (1999) 104(8) 125-31; Bodzioch et al., Nat. Genet. (1999) 22(4), 347-51; Orso et al., Nat. Genet. (2000) 24(2), 192-6).

Consequently, the presence of mutations or polymorphisms in the nucleotide sequence of ABCA1 appears to constitute, in the same way as the plasma concentration of HDL, a good risk factor for detecting a coronary disorder.

The high instance of coronary disease, which is a principal cause of mortality in developed countries, clearly shows the necessity of having tools for detecting allelic polymorphisms of the ABCA1 gene, and compositions, methods and kits which make it possible to diagnose the coronary risks, or a predisposition to a disease, linked to a circulating HDL deficiency, which would be likely to lead to angina pectoris, a myocardial infarction, atherosclerosis or a familial HDL deficiency such as Tangier disease, the symptoms of which are described above.

The human ABCA1 gene has recently been cloned and characterized and is described in applications PCT/FR00/01595 and EP99402668, and by Santamarina-Fojo et al., PNAS (2000) 97(14), 7987-7992). It has a total size of 149 kb and comprises a 1.453 kb promoter sequence, coding and intron sequences of 146.581 kb and a 1 kb 3′ region. It consists of 50 exons and, therefore, 49 introns. Exon 1 encodes the untranslated 5′ end (5′ UTR) and is followed by a large intron 24.156 kb in length. Exon 2 contains the remaining portion of the 5′ UTR end and encodes the first 21 amino acids of the N-terminal end of the ABCA1 protein. The ABCA1 gene encodes the 2261 amino acid ABCA1 transporter protein. Analysis of the regulatory sequence upstream of the human ABCA1 gene has made it possible to identify a TATA box (TCTATAAAAG) 33 bp upstream of the transcription start site, many binding sites for ubiquitous transcription factors such as SP1, NF-kB, and for activator proteins (AP-1, -2, and -4), and three E-box motifs (5′- CANNTG-3′), and several hepatocyte nuclear factor (HNF)-3 binding sites.

The applicants have discovered, surprisingly and unexpectedly, a number of polymorphisms in the human gene encoding the ABCA1 transporter. The applicants have, moreover, discovered a statistically significant correlation between the presence of certain allelic polymorphisms of the ABCA1 gene and the predisposition of an individual to develop a coronary pathology, such as a risk of myocardial infarction.

A subject of the present invention is therefore isolated nucleic acids encoding polymorphic variants of the human ABCA1 transporter, which can be linked to a coronary risk, such as a risk of myocardial infarction, a risk of cardiovascular disorder or more generally any disorder due to an HDL deficiency. A subject of the present invention is also the polymorphic polypeptide sequences of the human ABCA1 transporter, which are produced by the allelic forms of ABCA1. In addition, a subject of the present invention is recombinant vectors comprising these nucleic acids, cells comprising the vectors, and the methods for producing variant polypeptides obtained by cell culture under conditions which allow the expression of these polypeptides.

According to another aspect, a subject of the present invention is suitable detection means, probes or primers, specific for some of these polymorphisms in the human ABCA1 gene, which can be associated with coronary risks, such as for example a risk of myocardial infarction, or with a particular pharmacological response with respect to a therapeutic molecule. The present invention also relates to a method and kits which make it possible to detect a polymorphic sequence of the in the ABCA1 transporter gene of a human individual, and consists in (i) isolating a DNA sample from said individual, (ii) amplifying the regions containing the ABCA1 gene, (iii) determining the presence or absence of one or more polymorphisms in the amplified DNA region. Such polymorphisms can be linked to other polymorphisms in a polymorphic profile, which is associated with a predisposition to a metabolic disease or disorder.

Again according to another aspect, the present invention relates to a method which makes it possible to diagnose the risk of a cardiovascular disorder in a patient. The method consists in comparing two polymorphic profiles. The first profile is that of the patient the testing of whom is desired, and the second is a reference polymorphic profile which is derived from a population of control individuals with a predetermined cardiovascular or coronary risk such as, for example, a risk of myocardial infarction. The comparison between the test and control polymorphic sequences gives a good indication of the risk factor of the individual who has an ABCA1 polymorphic sequence.

GENERAL DEFINITIONS

The terms “nucleic acid” or “polynucleotide” refer to polymers containing purine and pyrimidine bases. They are polyribonucleotides, polydeoxyribonucleotides or polyribo-polydeoxyribonucleotide mixed polynucleotides. The nucleic acids comprise single- and double-stranded molecules, of the DNA-DNA, DNA-RNA, and RNA type, or protein nucleic acids (PNAs) formed by the conjugation of bases to an amino acid backbone. Nucleic acids also include molecules comprising modified bases and synthetic analogs of phosphodiester bonds, such as phosphorothioates and thioesters. The term “nucleic acid”, in particular DNA and RNA molecules, refers only to a primary or secondary structure of the molecule, and is not limited by a specific tertiary configuration. Consequently, the term includes double-stranded DNA, which includes, inter alia, linear or circular DNA molecules (including restriction fragments), plasmids and chromosomes. The structure of the double-stranded DNA molecules are described using the conventional direction, i.e., 5′->3′ of the nontranscribed strand of the DNA or sense strand (having a sequence homologous to the mRNA). A recombinant DNA molecule is a DNA molecule which has been subjected to molecular biological manipulations.

For the purposes of the present invention, the term an “isolated” nucleic acid or polypeptide is intended to mean a nucleic acid or polypeptide which has been removed from its environment of natural origin. An isolated nucleic acid or polypeptide contains less than approximately 50%, preferably less than 75%, and even more preferably less than 90% of cellular components.

A “derived” nucleic acid or polypeptide sequence corresponds to a region of a specific designated sequence, and includes, for the nucleic acids, the sequences which are identical or complementary.

For the purposes of the present invention, the expression “nucleotide sequence” can be used to designate either a polynucleotide or a nucleic acid, encompassing the genetic material itself, and is not, therefore, restricted to the information contained in the sequence.

For the purposes of the present invention, the term “oligonucleotide” refers to a nucleic acid which comprises generally at least 10, preferably 15, and more preferably at least 20 nucleotides, and which is capable of hybridizing to a genomic DNA molecule, to an mRNA or to a cDNA, or any other molecule of interest. Oligonucleotides can be labeled, for example with ³²P nucleotides or nucleotides to which are attached, by covalent bonding, a fluorescent label or a label such as biotin.

An oligonucleotide can be a probe capable of detecting the presence of a nucleic acid. Alternatively, the oligonucleotide can be a PCR primer, and can be used for cloning the complete sequence, or a fragment, of a gene of interest. An oligonucleotide can also form a triple helix with a double-stranded sequence of interest on a DNA molecule. Finally, an oligonucleotide library can be attached to a solid support in order to detect various polymorphisms of interest. Generally, oligonucleotides are synthetic and may comprise phosphodiester bonds or thioester bonds.

The term “probe” refers to a nucleic acid or to an oligonucleotide, capable of hybridizing to a sequence on a target nucleic acid due to the complementarity of at least a portion of the probe with a sequence of the target nucleic acid. Probes are generally labeled in order to be detectable after hybridization.

A nucleic acid molecule hybridizes to a nucleic acid molecule, such as a cDNA, a genomic DNA or an RNA, when the single-stranded form of the nucleic acid molecule can stabilize with another single-stranded nucleic acid molecule according to the appropriate conditions of temperature and ionic strength of the hybridization solution (Sambrook et al. 1989, Molecular Cloning: A Laboratory Manual, Second Edition, Cold Spring Harbor Laboratory Press: Cold Spring Harbor, N.Y.). The conditions of temperature and of ionic strength determine the stringency of the hybridization.

For preliminary screening of homologous nucleic acids, low stringency conditions (temperature equal to 55° C., 5×SSC containing 0.1% SDS, 0.25% milk, without formamide; or 30% formamide, 5×SSC containing 0.5% SDS) are used.

For moderate stringency hybridization conditions, a higher temperature is used, with 50% of formamide and 5×or 6×SSC. Although the hybridization reaction requires the nucleic acids to be complementary, mismatches between the bases are acceptable. The stringency suitable for the hybridization of a pair of nucleic acids depends on the length of the nucleic acids and on the degree of complementarity, which are variables well known in molecular biology. The greater the degree of similarity and of homology between two nucleotide sequences, the more a high hybridization temperature can be used. For hybrids with a size greater than about 100 nucleotides, the hybridization temperature is calculated using the equations described in Sambrook et al. For the hybridization of nucleic acids smaller in size, such as oligonucleotides, the position of the mismatches is important, and the length of the oligonucleotide determines its specificity. An oligonucleotide has, in general, a minimum length of 10 nucleotides, preferably at least 15 nucleotides, and even more preferably 20 nucleotides.

For high stringency hybridization conditions, the following conditions are used:

1- Competition of membranes and PREHYBRIDIZATION:

Mix: 40 μl of salmon sperm DNA (10 mg/ml)

+40 μl of human placental DNA (10 mg/ml)

Denature for 5 min at 96° C., then plunge the mixture into ice.

Remove the 2×SSC buffer and pour 4 ml of formamide mix into the hybridization tube containing the membranes.

Add the mixture of the two denatured DNAs.

Incubate at 42° C. for 5 to 6 hours, with rotation.

2- Competition of the labeled probe:

Add 10 to 50 μl of Cot I DNA, depending on the amount of nonspecific hybridization, to the labeled and purified probe.

Denature for 7 to 10 min at 95° C.

Incubate at 65° C. for 2 to 5 hours.

3- Hybridization:

Remove the prehybridization mix.

Mix 40 μl of salmon sperm DNA +40 μl of human placental DNA; denature for 5 min at 96° C., then plunge into ice.

Add 4 ml of formamide mix, the mixture of the two DNAs and the labeled probe/denatured Cot I DNA to the hybridization tube.

Incubate for 15 to 20 hours at 42° C., with rotation.

4- Washes:

One wash at room temperature in 2×SSC, to rinse.

Twice 5 minutes at room temperature, 2×SSC and 0.1% SDS

Twice 15 minutes, 0.1×SSC and 0.1% SDS, at 65° C.

Wrap the membranes in Saran wrap and expose to photographic emulsion.

The hybridization conditions described above are suitable for the hybridization, under high stringency conditions, of a nucleic acid molecule with a length which can range from 20 nucleotides to several hundreds of nucleotides.

The suitable hybridization conditions can, for example, be adjusted according to the teaching contained in the book by HAMES and HIGGINS (Eds., (1985) Nuclecic Acid hybridization, a practical approach, IRL Press, Oxford) or in the book by AUSUBEL et al (Current Protocols in Molecular Biology, Green Publishing Associates and Wiley Interscience, N.Y.).

A “gene” refers to a nucleic acid sequence corresponding to a sequnce present in the genome which comprises (i) the coding region, which comprises exons, introns and sequences at the junction between the exons and the introns,and (ii) regulatory sequences in 5′ and 3′ of the coding region.

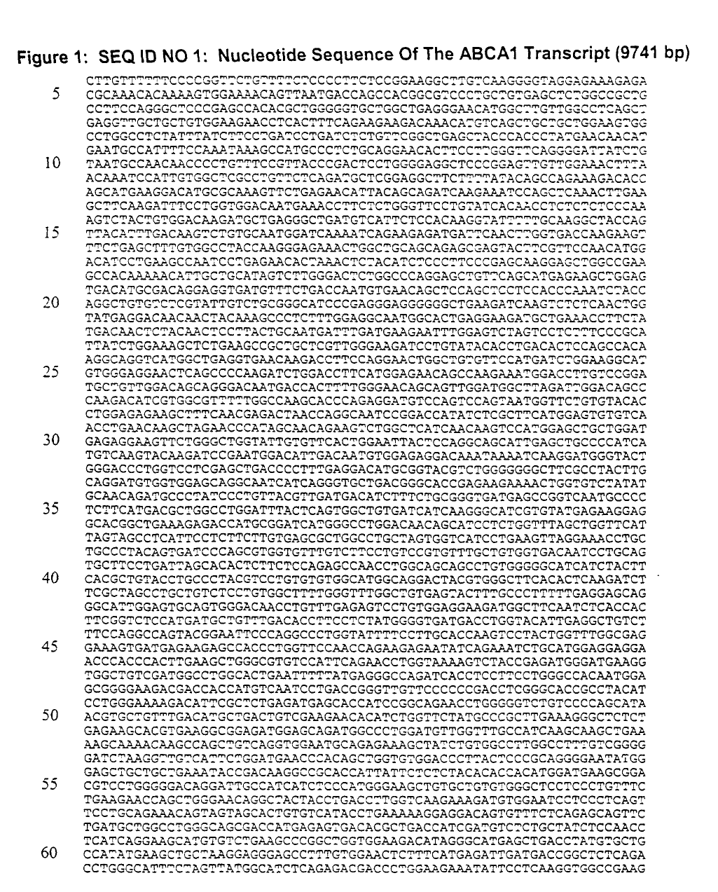

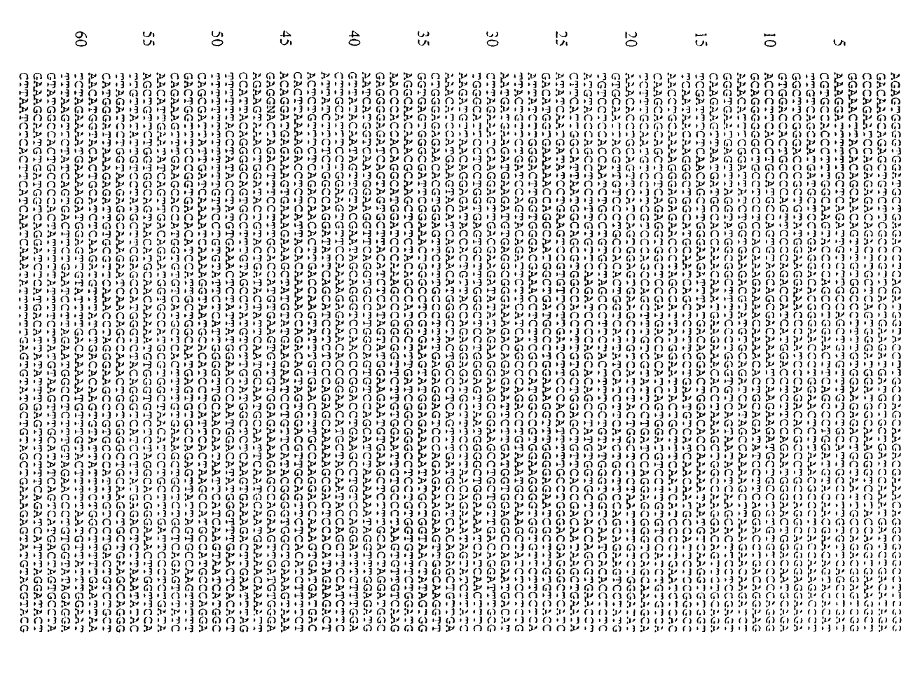

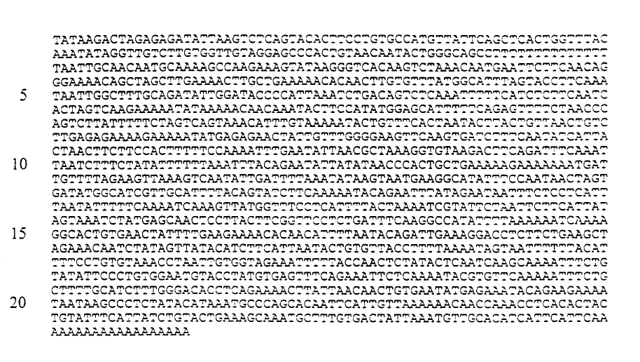

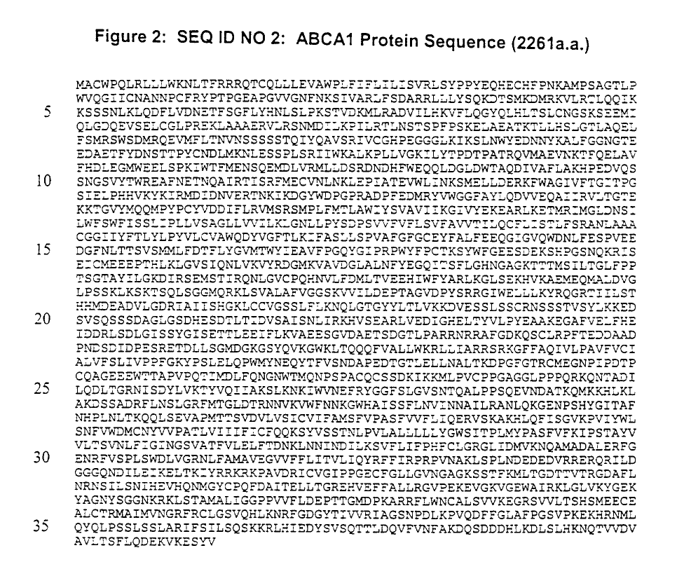

The term “ABCA1 transporter” is intended to mean the ABCA1 transporter protein encoded by the cDNA of nucleotide sequence SEQ ID NO: 1 (FIG. 1), and the peptide sequence SEQ ID NO: 2 (FIG. 2). In addition, several partial genomic nucleotide sequences of the ABCA1 gene have been isolated and characterized, these genomic sequences comprise both exon sequences and intron sequences. Some of these partial genomic sequences are given in Table 1, below.

TABLE 1
Partial genomic sequences of the human ABC1 gene

| SEQ ID NO | Designation |
|---|---|
| 3 | Promoter (p), exon 1a, intron 1a(p) |
| 4 | Intron 1a(p), exon 1b, intron 1b(p) |
| 5 | Intron 1b(p), exon 2, intron 2, exon 3, |
| | intron 3, exon 4, intron 4(p) |
| 6 | Intron 4(p), exon 5, intron 5(p) |
| 7 | Intron 5(p), exon 6, intron 6(p) |
| 8 | Intron 7(p), exon 8, intron 8(p) |
| 9 | Intron 8(p), exon 9, intron 9, exon 10, |
| | intron 10(p) |
| 10 | Intron 11(p), exon 12, intron 12, exon 13, |
| | intron 13, exon 14, intron 14, exon 15, |
| | intron 15, exon 16, intron 16, exon 17, |
| | intron 17(p) |
| 11 | Intron 17(p), exon 18, intron 18(p) |
| 12 | Intron 18(p), exon 19, intron 19(p) |
| 13 | Intron 19(p), exon 20, intron 20, exon 21, |
| | intron 21, exon 22, intron 22, exon 23, |
| | intron 23, exon 24, intron 24, exon 25, |
| | intron 25, exon 26, intron 26(p) |
| 14 | Intron 26(p), exon 27, intron 27, exon 28, |
| | intron 28, exon 29, intron 29, exon 30, |
| | intron 30(p) |
| 15 | Intron 30(p), exon 31, intron 31, exon 32, |
| | intron 32, exon 33, intron 33, exon 34, |
| | intron 34(p) |
| 16 | Intron 35(p), exon 36, intron 36(p) |
| 17 | Intron 36(p), exon 37, intron 37, exon 38, |
| | intron 38(p) |
| 18 | Intron 38(p), exon 39, Intron 39(p) |
| 19 | Intron 39(p), exon 40, intron 40, exon 41, |
| | intron 41(p) |
| 20 | Intron 44(p), exon 45, intron 45(p) |
| 21 | Intron 45(p), exon 46, intron 46, exon 47, |
| | intron 47(p) |
| 22 | Intron 48(p), exon 49, sequence in 3′ of last exon |

The expression “single nucleotide polymorphism” or “SNP” is intended to mean the substitution, insertion or deletion of a single nucleotide in the nucleotide sequence of a gene, among several individuals. When the polymorphism is in the form of an insertion or of a deletion, it may be an insertion or a deletion of one or more nucleotides at one position of a gene. The various nucleotide sequences of the same gene that result from such polymorphisms are alleles.

A “polymorphic position” is a predetermined position in the sequence of agene which comprises a SNP. In certain cases, genetic polymorphisms cause a variation in the amino acid sequence and, therefore, from a polymorphic position can result in a polymorphism in the amino acid sequence at a predetermined position of the sequence of the polypeptide. An individual homozygous for a specific polymorphism carries, on the two copies of the same gene, the same polymorphic sequence at the same position. An individual heterozygous for a specific polymorphism carries, on the two copies of the gene, sequences which are different at the polymorphic position.

The term “polymorphic profile” is intended to mean one or more single nucleotide polymorphisms, which can be present on the sequence of a single gene or of a plurality of genes. A simple polymorphic profile comprises a SNP in a single position of one or two alleles of an individual (allelic polymorphism). A test polymorphic profile corresponds to the characteristic polymorphism of an individual who is diagnosed, for example, with a predisposition to a disease associated with a deficiency of the ABCA1 gene, such as a metabolic deficiency of the FHD type or a cardiovascular disorder, or a risk of a myocardial infarction. The reference or control polymorphic profile was determined by statistically significant correlation of the profiles in a population of individuals who exhibit a coronary risk.

POLYMORPHISMS OF THE ABCA1 GENE

The present invention is based on the discovery of 90 polymorphisms in the sequence of the human ABCA1 gene, as represented in the sequences SEQ ID NOS: 3-22, of which 22 SNPs are found in the promoter sequence upstream of the human ABCA1 gene of SEQ ID NO: 23. Some SNPs according to the present invention lead to structural modifications in the polypeptide sequence of the human ABCA1 transporter protein represented in SEQ ID NO: 2.

For the identification and characterization of ABCA1 polymorphic DNA, polymerase chain reactions (PCRs) were carried out in order to amplify the ABCA1 sequences from human genomic DNA originating from three different ethnic groups, namely, a Caucasian population, a Japanese population and/or an African population. The PCR products were sequenced, and the sequences were compared with one another and with the reference sequences SEQ ID NOS: 3-23.

Table 2 gives the allelic polymorphisms in the exons and introns of the human ABCA1 gene according to the present invention. It indicates in particular the polymorphic position in each of the exons with respect to the first nucleotide of the reference exon, and in each of the introns with respect to the 3′ end or upstream of the 5′ end of the closest exon, the 5′ and 3′ sequences on either side of the polymorphic position, and the frequency of appearance of the polymorphism in the three populations tested, African, Caucasian and Japanese. In cases in which these frequencies could not be determined, “n.d.” is indicated. Some polymorphisms cause structural modifications in the peptide sequence of the human ABCA1 transporter protein, and, more precisely, cause the substitution of one amino acid with another (MIS). Other polymorphisms in the exon sequences are termed “silent” (SIL), the ABCA1 protein sequence remaining unchanged. Finally, when the polymorphism is located in an intron or in a noncoding exon, the polymorphic sequence is termed “noncoding” (NCD). The polymorphisms identified in the various exons and introns of the ABCA1 gene are described in detail below.

TABLE 2
Allelic Polymorphisms In The Exon And Introns Of The Human ABCA1 Gene

| | | | | | | | | Pro- | | Frequency In The Population | | |
|---|---|---|---|---|---|---|---|---|---|---|---|---|
| | Intron/ | Posi- | | Allele | Allele | | | tein | | | Cau- | |
| Name | Exon | tion | 5′-Sequence | 1 | 2 | 3′-Sequence | Type | Change | Codon | African | casian | Japanese |
| s-35e1a | Exon 1a | 35 | GGCCGGGACC | C | G | GCAGAGCCGA | NCD | | 0 | 0.00% | 4.84% | 0.00% |
| s-16e1b | Exon 1b | 16 | GACCAGCCAC | — | G | GGCGTCCCTG | NCD | | 0 | 0.00% | 10.94 | 27.42% |
| s-76e1b | Exon 1b | 76 | ACACGCTGGG | G | C | GTGCTGGCTG | NCD | | 0 | 0.00% | 370.00% | 15.00% |
| s-m39i3 | Intron | −39 | GGCAGTTGGC | C | A | TAGCTAAAGC | NCD | | 0 | 21.67% | 0.00% | 0.00% |
| | 3 | | | | | | | | | | | |
| s-25i4 | Intron | 25 | TCTCTGCATC | C | T | GTTGAGAATG | NCD | | 0 | 6.90% | 0.00% | 0.00% |
| | 4 | | | | | | | | | | | |
| s-53e5 | Exon 5 | 53 | CTGGGTTCCT | G | A | TATCACAACC | SIL | | 0 | 36.67% | 26.56% | 62.90% |
| s-108e6 | Exon 6 | 108 | TTTGTGGCCT | A | G | CCAAGGGAGA | SIL | | 217 | 3.45% | 0.00% | 0.00% |
| s-113e6 | Exon 6 | 113 | GGCCTACCAA | G | A | GGAGAAACTG | MIS | R>K | 219 | 61.67% | 20.37% | 46.55% |
| s-m14i7 | Intron | −14 | TTCTGTCCCC | — | A | ATCCCTGACG | NCD | | 0 | 16.67% | 12.07% | 63.33% |
| | 7 | | | | | | | | | | | |
| s-123e8 | Exon 8 | 123 | GCGGGCATCC | C | T | GAGGGAGGGG | SIL | | 312 | 12.07% | 6.67% | 10.34% |
| s-135e8 | Exon 8 | 135 | AGGGAGGGGG | G | A | CTGAAGATCA | SIL | | 316 | 32.76% | 15.00% | 6.90% |
| s-m89i8 | Intron | −89 | TGGGGTTTCA | A | G | CTAAGAACTC | NCD | | 0 | 23.33% | 0.00% | 0.00% |
| | 8 | | | | | | | | | | | |
| s-m104i9 | Intron | −104 | GAGGACTGGC | A | G | CAGGGCTGCT | NCD | | 0 | n.d. | 14.81% | 28.85% |
| | 9 | | | | | | | | | | | |
| s-m61i9 | Intron | −61 | AGGAGCCAAA | — | G | CGCTCATTGT | NCD | | 0 | n.d. | 39.58% | 0.00% |
| | 9 | | | | | | | | | | | |
| s-m41i9 | Intron | −41 | GTCTGTGCTT | CTC | - - - | CTCCTTTTTC | NCD | | 0 | n.d. | 16.00% | 0.00% |
| | 9 | | | | | | | | | | | |
| s-m13i9 | Intron | −13 | CCTGGCTCCC | C | T | ACCTGACTCC | NCD | | 0 | n.d. | 8.00% | 42.86% |
| | 9 | | | | | | | | | | | |
| s-126e12 | Exon 12 | 126 | CTCCAGGCAG | C | T | AATTGAGCTG | SIL | | 545 | 9.26% | 0.00% | 0.00% |
| s-m29i12 | Intron | −29 | TTCTAAAGGA | A | G | TGGTTGATTA | NCD | | 0 | 6.00% | 0.00% | 0.00% |
| | 12 | | | | | | | | | | | |
| s-24i13 | Intron | 24 | AAGCCACTGT | T | A | TTTAACCAGT | NCD | | 0 | 5.36% | 26.56% | 12.90% |
| | 13 | | | | | | | | | | | |
| s-m59i13 | Intron | −59 | GGCCTTGGCC | C | T | ATCACCCTGG | NCD | | 0 | 0.00% | 7.81% | 34.38% |
| | 13 | | | | | | | | | | | |
| s-148e14 | Exon 14 | 148 | ACAACAGCAT | C | A | CTCTGGTTTA | SIL | | 680 | 50.00% | 13.46% | 71.88% |
| s-81i14 | Intron | 81 | AAGAAGAAAA | G | A | AAATCCAAGC | NCD | | 0 | 0.00% | 0.00% | 32.81% |
| | 14 | | | | | | | | | | | |
| s-115i14 | Intron | 115 | GGGGTCATAC | C | A | TGTCATTTCC | NCD | | 0 | 0.00% | 0.00% | 34.38% |
| | 14 | | | | | | | | | | | |
| s-196e15 | Exon 15 | 196 | GCAGGACTAC | G | A | TGGGCTTCAC | MIS | V>M | 771 | 5.36% | 1.72% | 6.67% |
| s-136e16 | Exon 16 | 136 | CACCACTTCG | G | A | TCTCCATGAT | MIS | V>I | 825 | 0.00% | 6.25% | 37.50% |
| s-27e17 | Exon 17 | 27 | CAGGCCCTGG | T | C | ATTTTCCTTG | MIS | Y>P | 857 | 0.00% | 11.11% | 0.00% |
| s-107e17 | Exon 17 | 107 | AGAAGAGAAT | A | G | TCAGAAAGTA | MIS | I>M | 883 | 45.65% | 12.90% | 71.43% |
| s-60i17 | Intron | 60 | CTGCTAACTG | T | C | TGGGGGCAAG | NCD | | 0 | 10.87% | 0.00% | 0.00% |
| | 17 | | | | | | | | | | | |
| s-m68i17 | Intron | −68 | AGCGCAGTGC | C | G | CTGTGTCCTT | NCD | | 0 | 0.00% | 8.33% | 32.81% |
| | 17 | | | | | | | | | | | |
| s-4e18 | Exon 18 | 4 | GTCACAGTCT | G | T | CATGGAGGAG | MIS | C>F | 887 | 0.00% | 0.00% | 3.13% |
| s-40e19 | Exon 19 | 40 | CCTCGGGCAC | C | T | GCCTACATCC | SIL | | 956 | 0.00% | 0.00% | 4.35% |
| s-18i19 | Intron | 18 | CCAGCAGCAC | G | A | TTAAGAATAG | NCD | | 0 | 15.79% | 0.00% | 0.00% |
| | 19 | | | | | | | | | | | |
| s-m10i19 | Intron | −10 | ACCTGCCTCC | C | T | TGTCCCCAGG | NCD | | 0 | 4.55% | 0.00% | 0.00% |
| | 19 | | | | | | | | | | | |
| s-123e22 | Exon 22 | 123 | GAAGAACCAG | C | T | TGGGAACAGG | MIS | L>C | 1122 | 29.31% | 0.00% | 0.00% |
| s-m65i22 | Intron | −65 | CCTCCCCGGC | G | A | AAGGTGCTGG | NCD | | 0 | 0.00% | 6.45% | n.d. |
| | 22 | | | | | | | | | | | |
| s-54e23 | Exon 23 | 54 | GCGACCATGA | G | C | AGTGACACGC | MIS | E>D | 1172 | 7.14% | 4.84% | n.d. |
| s-149i23 | Exon 23 | 149 | GGTGTGGGAG | C | T | AGCTTGGTGG | NCD | | 0 | 7.14% | 0.00% | n.d. |
| s-m50i23 | Intron | −50 | GCCCTCACTC | C | T | GAAGCCCCTC | NCD | | 0 | 3.45% | 0.00% | 0.00% |
| | 23 | | | | | | | | | | | |
| s-98e24 | Exon 24 | 98 | TGCCATATGA | A | G | GCTGCTAAGG | SIL | | 1211 | 3.45% | 0.00% | 0.00% |
| s-149e24 | Exon 24 | 149 | TTGATGACCG | G | A | CTCTCAGACC | SIL | | 1228 | 18.97% | 0.00% | 0.00% |
| s-44e27 | Exon 27 | 44 | ATGGCAAAGG | G | A | TCCTACCAGG | SIL | | 1315 | 11.54% | 0.00% | n.d. |
| s-7e30 | Exon 30 | 7 | GCAGAGACAC | G | A | CCCTGCCAGG | SIL | | 1427 | 3.45% | 12.50% | 34.48% |
| s-166e30 | Exon 30 | 166 | GTCCCCCAGG | G | T | GCAGGGGGGC | SIL | | 1480 | 6.90% | 0.00% | 0.00% |
| s-30i31 | Intron | 30 | GGCCCCTGCC | T | G | TATTATTACT | NCD | | 0 | 13.79% | 12.50% | 0.00% |
| | 31 | | | | | | | | | | | |
| s-m142i32 | Intron | −142 | AGGAAGCTGA | C | T | GTGTAACTTC | NCD | | 0 | 5.36% | 0.00% | 0.00% |
| | 32 | | | | | | | | | | | |
| s-m130i32 | Intron | −130 | TGTAACTTCT | -- | CT | GAGGCAAAAT | NCD | | 0 | 0.00% | 26.79% | 40.00% |
| | 32 | | | | | | | | | | | |
| s-m26i32 | Intron | −26 | CACTGTCTGG | G | C | TTTTAATGTC | NCD | | 0 | 3.57% | 8.93% | 18.33% |
| | 32 | | | | | | | | | | | |
| s-m74i33 | Intron | −74 | CCTGTTGTCC | T | A | CAGGTTCCAG | NCD | | 0 | 3.45% | 0.00% | 0.00% |
| | 33 | | | | | | | | | | | |
| s-62e34 | Exon 34 | 62 | CTGGACACCA | G | A | AAATAATGTC | NIS | R>K | 1587 | 93.10% | 26.67% | 57.14% |
| s-77e36 | Exon 36 | 77 | CAGCTTTGTC | G | A | TATTCCTGAT | NIS | V>I | 1674 | 0.00% | 0.00% | 3.33% |
| s-55i36 | Intron | 55 | CATGGATTGA | T | C | ATGGATAAGA | NCD | | 0 | 24.14% | 0.00% | 0.00% |
| | 36 | | | | | | | | | | | |
| s-64e38 | Exon 38 | 64 | GCACAGCCTA | T | C | GTGGTGCTCA | SIL | | 1767 | 4.00% | 0.00% | n.d. |
| s-251i39 | Intron | 251 | GAAAATAGTG | T | G | TATTTGCTTG | NCD | | 0 | n.d. | 24.14% | 46.55% |
| | 39 | | | | | | | | | | | |
| s-252i39 | Intron | 252 | AAAATAGTGT | T | C | ATTTGCTTGG | NCD | | 0 | n.d. | 24.14% | 44.83% |
| | 39 | | | | | | | | | | | |
| s-m119i40 | Intron | −119 | ATCCCGCAGC | C | T | CCCTCCCTGC | NCD | | 0 | 0.00% | 0.00% | 25.00% |
| | 40 | | | | | | | | | | | |
| s-m69i44 | Intron | −69 | CTTTCATACA | T | C | GTATGTGTAG | NCD | | 0 | 5.17% | 0.00% | n.d. |
| | 44 | | | | | | | | | | | |
| s-114e45 | Exon 45 | 114 | CTTTGATCGG | C | T | GGGCCTCCTG | SIL | | 2061 | 22.41% | 0.00% | n.d. |
| s-m34i45 | Intron | −34 | TTTTTTCCCC | C | T | CAGCAAATAA | NCD | | 0 | n.d. | 5.17% | 0.00% |
| | 45 | | | | | | | | | | | |
| s-13i47 | Intron | 13 | AATAAAGATA | A | G | TTTCTTTGGG | NCD | | 0 | 0.00% | 11.67% | 42.86% |
| | 47 | | | | | | | | | | | |
| s-86i47 | Intron | 86 | GTGCCTCTAA | A | C | ATAAAGGGAA | NCD | | 0 | 1.72% | 13.33% | 0.00% |
| | 47 | | | | | | | | | | | |
| s-377e49 | Exon 49 | 377 | GACTTGAATT | T | C | AGTTTTTTAC | NCD | | 0 | 3.33% | 0.00% | 0.00% |
| s-833e49 | Exon 49 | 833 | TGGGTGTCTC | C | T | AGGCACGGGA | NCD | | 0 | 54.55% | 35.00% | 18.97% |
| s-1580e49 | Exon49 | 1580 | TGTAACAATA | C | T | TGGGCAGCCT | NCD | | 0 | 0.00% | 30.65% | 0.00% |
| s-1791e49 | Exon49 | 1791 | AGTCTCAAAT | A | T | TTTCATCTCT | NCD | | 0 | 15.79% | 0.00% | 0.00% |
| s-2034e49 | Exon49 | 2034 | ACTTTTTCCA | A | G | AATTTGAATA | NCD | | 0 | 3.33% | 12.90% | 22.22% |
| s-2049e49 | Exon 49 | 2049 | TGAATATTAA | C | T | GCTAAAGGTG | NCD | | 0 | 30.00% | 0.00% | 24.14% |
| s-2449e49 | Exon 49 | 2449 | AAGAAAACAC | A | G | ACATTTTAAT | NCD | | 0 | 16.67% | 20.00% | 23.33% |
| s-2843e49 | Exon 49 | 2843 | TTCATTATCT | G | A | TACTGAAAGC | NCD | | 0 | 7.69% | 0.00% | 0.00% |

Mutation in exon 1a

The mutation s-35e1a consists of the substitution of a cytosine (C) with a guanine (G) at position 35 of exon 1a, i.e., at position 2928 with respect to the sequence SEQ ID NO: 3. The exon 1a carrying this polymorphism has the nucleotide sequence SEQ ID NO: 24. The cDNA carrying the polymorphism in exon 1a of the ABCA1 gene is represented by the nucleotide sequence SEQ ID NO: 92.

Mutations in exon 1b

A first mutation is designated s-16e1b and consists of the insertion of a guanine (G) at position 16 of exon 1b, i.e., at position 115 with respect to the sequence SEQ ID NO: 4. The exon 1b carrying this polymorphism has the nucleotide sequence SEQ ID NO: 25. The cDNA carrying the polymorphism in exon 1b of the ABCA1 gene is represented by the nucleotide sequence SEQ ID NO: 93.

A second mutation designated s-76e1b consists of the substitution of a guanine (G) with a cytosine (C) at position 76 of exon 1b, i.e., at position 175 with respect to the sequence SEQ ID NO: 4. The exon 1b carrying this polymorphism has the nucleotide sequence SEQ ID NO: 26. The cDNA carrying the polymorphism in exon 1b of the ABCA1 gene is represented by the nucleotide sequence SEQ ID NO: 94.

Mutation in intron 3

The mutation s-m39i3 consists of the substitution of a cytosine (C) to to an adenine (A) [lacuna] position −39 of the 3′ end of intron 3, i.e., at position 10646 with respect to the sequence SEQ ID NO: 5. The nucleotide sequence of the intron 3 carrying this polymorphism is represented in the sequence SEQ ID NO: 27.

Mutation in intron 4

The mutation s-25i4 consists of the substitution of a cytosine (C) to a thymidine (T) [lacuna] position 25 of the 5′ end of intron 4, i.e., at position 10828 with respect to the sequence SEQ ID NO: 5. The partial nucleotide sequence of the intron 4 carrying this polymorphism is represented in the sequence SEQ ID NO: 28.

Mutation in exon 5

The mutation s-53e5 consists of the substitution of a guanine (G) to an adenine (A) at position 53 of exon 5, i.e., at position 306 with respect to the sequence SEQ ID NO: 6. The exon 5 carrying this polymorphism has the nucleotide sequence SEQ ID NO: 29. The cDNA carrying the polymorphism in exon 5 of the ABCA1 gene is represented by the nucleotide sequence SEQ IN NO: 95.

Mutations in exon 6

The mutation designated s-108e6 consists of the substitution of an adenine (A) to a guanine (G) at position 108 of exon 6, i.e., at position 268 with respect to the sequence SEQ ID NO: 7. The exon 6 carrying this polymorphism has the nucleotide sequence SEQ ID NO: 30. The cDNA carrying the polymorphism in exon 6 of the ABCA1 gene is represented by the nucleotide sequence SEQ ID NO: 96.

The mutation s-113e6 consists of the substitution of a guanine (G) to an adenine (A) at position 113 of exon 6, i.e., at position 273 with respect to the sequence SEQ ID NO: 7. The sequence of exon 6 of the ABCA1 gene carrying this polymorphism is the polynucleotide of sequence SEQ ID NO: 31. The mutated cDNA carrying the polymorphism in exon 6 is represented in the nucleotide sequence SEQ ID NO: 97, encodes a variant ABCA1 polypeptide 2261 amino acids in length, of sequence SEQ ID NO: 127.

Mutation in intron 7

The mutation s-m14i7 consists of the insertion of an adenine (A) at position -14 upstream of the 3′ end of intron 7, i.e., at position 589 with respect to the sequence SEQ ID NO: 8. The partial nucleotide sequence of the intron 7 carrying this polymorphism is represented in the sequence SEQ ID NO: 32.

Mutations in exon 8

The mutation s-123e8 consists of the substitution of a cytosine (C) with a thymine (T) at position 123 of exon 8, i.e., at position 726 with respect to the sequence SEQ ID NO: 8. The exon 8 carrying this polymorphism has the nucleotide sequence SEQ ID NO: 33. The cDNA carrying the polymorphism in exon 8 of the ABCA1 gene is represented by the nucleotide sequence SEQ ID NO: 98.

The mutation s-135e8 consists of the substitution of a guanine (G) with an adenine (A) at position 135 of exon 8, i.e., at position 738 with respect to the sequence SEQ ID NO: 8. The exon 8 carrying this polymorphism has the nucleotide sequence SEQ ID NO: 34. The cDNA carrying the polymorphism in exon 8 of the ABCA1 gene is represented by the nucleotide sequence SEQ ID NO: 99.

Mutation in intron 8

The mutation s-m89i8 consists of the substitution of an adenine (A) with a guanine (G) at position -89 upstream of the 3′ end of intron 8, i.e., at position 525 with respect to the sequence SEQ ID NO: 9. The partial nucleotide sequence of the intron 8 carrying this polymorphism is represented in the nucleotide sequence SEQ ID NO: 35.

Mutations in intron 9

The mutation s-m104i9 consists of the substitution of an adenine (A) with a guanine (G) at position −104 upstream of the 3′ end of intron 9, i.e., at position 981 with respect to the sequence SEQ ID NO: 9. The partial nucleotide sequence of the intron 9 carrying this polymorphism is represented in the nucleotide sequence SEQ ID NO: 36.

The mutation s-m61 i9 consists of an insertion of a guanine (G) at position -61 upstream of the 3′ end of intron 9, i.e., at position 1023 with respect to the sequence SEQ ID NO: 9. The partial nucleotide sequence of the intron 9 carrying this polymorphism is represented in the sequence SEQ ID NO: 37.

The mutation s-m41 i9 consists of a deletion of a three-nucleotide “CTC” fragment from the nucleotide at position −41 to nucleotide -43 upstream of the 3′ end of intron 9, i.e., at position 1042-4044 with respect to the sequence SEQ ID NO: 9. The partial nucleotide sequence of the intron 9 carrying this polymorphism is represented in the nucleotide sequence SEQ ID NO: 38.

The mutation s-m13i9 consists of the substitution of a cytosine (C) with a thymine (T) at position -13 upstream of the 3′ end of intron 9, i.e., at position 1072 with respect to the sequence SEQ ID NO: 9. The partial nucleotide sequence of the intron 9 carrying this polymorphism is represented in the nucleotide sequence SEQ ID NO: 39.

Mutation in exon 12

The mutation s-126e12 consists of the substitution of a cytosine (C) to a thymine (T) at position 126 of exon 12, i.e., at position 765 with respect to the sequence SEQ ID NO: 10. The exon 12 carrying this polymorphism has the nucleotide sequence SEQ ID NO: 40. The cDNA carrying the polymorphism in exon 12 of the ABCA1 gene is represented by the nucleotide sequence SEQ ID NO: 100.

Mutation in intron 12

The mutation s-m29i12 consists of the substitution of an adenine (A) with a guanine (G) at position −29 upstream of the 3′ end of intron 12, i.e., at position 1337 with respect to the sequence SEQ ID NO: 10. The nucleotide sequence of the intron 12 carrying this polymorphism is represented in the sequence SEQ ID NO: 41.

Mutations in intron 13

The mutation s-24i1 3 consists of the substitution of a thymine (T) with an adenine (A) at position 24 of the 5′ end of intron 13, i.e., at position 1566 with respect to the sequence SEQ ID NO: 10. The nucleotide sequence of the intron 13 carrying this polymorphism is represented in the nucleotide sequence SEQ ID NO: 42.

The mutation s-m59i1 3 consists of the substitution of a cytosine (C) with a thymine (T) at position −59 upstream of the 3′ end of intron 13, i.e., at position 3270 with respect to the sequence SEQ ID NO: 10. The nucleotide sequence of the intron 13 carrying this polymorphism has the sequence SEQ ID NO: 43.

Mutation in exon 14

The mutation s-148e14 consists of the substitution of a cytosine (C) with an adenine (A) at position 148 of exon 14, i.e., at position 3476 with respect to the sequence SEQ ID NO: 10. The exon 14 carrying this polymorphism has the nucleotide sequence SEQ ID NO: 44. The cDNA carrying the polymorphism in exon 14 of the ABCA1 gene is represented by the nucleotide sequence SEQ ID NO: 101.

Mutations in intron 14

The mutation s-81i14 consists of the substitution of a guanine (G) with an adenine (A) at position 81 of the 5′ end of intron 14, i.e., at position 3632 with respect to the sequence SEQ ID NO: 10. The nucleotide sequence of the intron 14 carrying this polymorphism is represented in the sequence SEQ ID NO: 45.

The mutation s-115i14 consists of the substitution of a cytosine (C) with an adenine (A) at position 115 of the 5′ end of intron 14, i.e., at position 3666 with respect to the sequence SEQ ID NO: 10. The intron 14 carrying this polymorphism has the nucleotide sequence SEQ ID NO: 46.

Mutation in exon 15

The mutation s-196e15 consists of the substitution of a guanine (G) with an adenine (A) at position 196 of exon 15, i.e., at position 5492 with respect to the sequence SEQ ID NO: 10. The exon 15 carrying this polymorphism has the nucleotide sequence SEQ ID NO: 47. The cDNA carrying the polymorphism in exon 15 of the ABCA1 gene is represented by the nucleotide sequence SEQ ID NO: 102 and encodes a mutated ABCA1 protein SEQ ID NO: 128 in which a valine (V) is substituted with a methionine (M) at position 771.

Mutation in exon 16

The mutation s-136e16 consists of the substitution of a guanine (G) with an adenine (A)at position 136 of exon 16, i.e., at position 6714 with respect to the sequence SEQ ID NO: 10. The exon 16 carrying this polymorphism has the nucleotide sequence SEQ ID NO: 48. The cDNA carrying the polymorphism in exon 16 of the ABCA1 gene is represented by the nucleotide sequence SEQ ID NO: 103 and encodes a mutated ABCA1 protein of sequence SEQ ID NO: 129, which comprises the substitution of a valine (V) to isoleucine (I) at position 825.

Mutations in exon 17

The mutation s-27e17 consists of the substitution of a thymine (T) with a cytosine (C) at position 27 of exon 17, i.e., at position 7914 with respect to the sequence SEQ ID NO: 10. The exon 17 carrying this polymorphism has the nucleotide sequence SEQ ID NO: 49. The cDNA carrying the polymorphism in exon 17 of the ABCA1 gene is represented by the nucleotide sequence SEQ ID NO: 104 and encodes a mutated ABCA1 protein SEQ ID NO: 130 in which a tyrosine (Y) is substituted with a proline (P) at position 857.

The mutation s-107e17 consists of the substitution of an adenine (A) with a guanine (G) at position 107 of exon 17, i.e., at position 7994 with respect to the sequence SEQ ID NO: 10. The exon 17 carrying this polymorphism has the nucleotide sequence SEQ ID NO: 50. The cDNA carrying the polymorphism in exon 17 of the ABCA1 gene is represented by the nucleotide sequence SEQ ID NO: 105 and encodes a mutated ABCA1 protein SEQ ID NO: 131 in which an isoleucine (I) is substituted with a methionine (M) at position 883.

Mutations in intron 17

The mutation s-60i17 consists of the substitution of a thymine (T) with a cytosine (C) at position 60 of the 5′ end of intron 17, i.e., at position 8061 with respect to the sequence SEQ ID NO: 10. The partial nucleotide sequence of the intron 17 carrying this polymorphism is represented in the sequence SEQ ID NO: 51.

The mutation s-m68i17 consists of the substitution of a cytosine (C) with a guanine (G) at position −68 upstream of the 3′ end of intron 17, i.e., at position 319 with respect to the sequence SEQ ID NO: 11. The partial nucleotide sequence of the intron 17 carrying this polymorphism is represented in the sequence SEQ ID NO: 52.

Mutation in exon 18

The mutation s-4e18 consists of the substitution of a guanine (G) with a thymine (T) at position 4 of exon 18, i.e., at position 390 with respect to the sequence SEQ ID NO: 11. The exon 18 carrying this polymorphism has the nucleotide sequence SEQ ID NO: 53. The cDNA carrying the polymorphism in exon 18 of the ABCA1 gene is represented by the nucleotide sequence SEQ ID NO: 106 and encodes a mutated ABCA1 protein SEQ ID NO: 132 in which a cysteine (C) is substituted with a phenylalanine (F) at position 887.

The mutation s-40e19 consists of the substitution of a cytosine (C) with a thymine (T) at position 40 of exon 19, i.e., at position 943 with respect to the sequence SEQ ID NO: 12. The exon 19 carrying this polymorphism has the nucleotide sequence SEQ ID NO: 54. The cDNA carrying the polymorphism in exon 19 of the ABCA1 gene is represented by the nucleotide sequence SEQ ID NO: 107.

Mutations in intron 19

The mutation s-18i19 consists of the substitution of a guanine (G) with an adenine (A) at position 18 of the 5′ end of intron 19, i.e., at position 1053 with respect to the sequence SEQ ID NO: 12. The partial nucleotide sequence of the intron 19 carrying this polymorphism is represented in the sequence SEQ ID NO: 55.

The mutation s-m10i19 consists of the substitution of a cytosine (C) with a thymine (T) at position −10 upstream of the 3′ end of intron 19, i.e., at position 274 with respect to the sequence SEQ ID NO: 13. The partial nucleotide sequence of the intron 19 carrying this polymorphism is represented in the sequence SEQ ID NO: 56.

Mutation in exon 22

The mutation s-123e22 consists of the substitution of a cytosine (C) with a thymine (T) at position 123 of exon 22, i.e., at position 1592 with respect to the sequence SEQ ID NO: 13. The exon 22 carrying this polymorphism has the nucleotide sequence SEQ ID NO: 57. The cDNA carrying the polymorphism in exon 22 of the ABCA1 gene is represented by the nucleotide sequence SEQ ID NO: 108 and encodes a mutated ABCA1 protein SEQ ID NO: 133 in which a leucine (L) is substituted with a cysteine (C) at position 1122.

Mutation in intron 22

The mutation s-m65i22 consists of the substitution of a guanine (G) with an adenine (A) at position −65 upstream of the 3′ end of intron 22, i.e., at position 2884 with respect to the sequence SEQ ID NO: 13. The nucleotide sequence of intron 22 carrying this polymorphism is represented in the sequence SEQ ID NO: 58.

Mutation in exon 23

The mutation s-54e23 consists of the substitution of a guanine (G) with a cytosine (C) at position 54 of exon 23, i.e., at position 3002 with respect to the sequence SEQ ID NO: 13. The exon 23 carrying this polymorphism has the nucleotide sequence SEQ ID NO: 59. The cDNA carrying the polymorphism in exon 23 of the ABCA1 gene is represented by the nucleotide sequence SEQ ID NO: 109, and encodes a mutated ABCA1 protein SEQ ID NO: 134 in which a glutamic acid (E) is substituted with an aspartic acid (D) at position 1172.

Mutations in intron 23

The mutation s-149i23 consists of the substitution of a cytosine (C) with a thymine (T) at position 149 of the 5′ end of intron 23, i.e., at position 3170 with respect to the sequence SEQ ID NO: 13. The nucleotide sequence of the intron 23 carrying this polymorphism is represented in the sequence SEQ ID NO: 60.

The mutation s-m50i23 consists of the substitution of a cytosine (C) with a thymine (T) at position -50 upstream of the 3′ end of intron 23, i.e., at position 3958 with respect to the sequence SEQ ID NO: 13. The sequence of the intron 23 carrying this polymorphism is represented in the nucleotide sequence SEQ ID NO: 61.

Mutations in exon 24

The mutation s-98e24 consists of the substitution of an adenine (A) with a guanine (G) at position 98 of exon 24, i.e., at position 4105 with respect to the sequence SEQ ID NO: 13. The exon 24 carrying this polymorphism has the nucleotide sequence SEQ ID NO: 62. The cDNA corresponding to the mutation in exon 24 of the ABCA1 gene is represented by the nucleotide sequence SEQ ID NO: 110.

The mutation s-149e24 consists of the substitution of a guanine (G) with an adenine (A) at position 149 of exon 24, i.e., at position 4156 with respect to the sequence SEQ ID NO: 13. The exon 24 carrying this polymorphism has the nucleotide sequence SEQ ID NO: 63. The cDNA carrying the polymorphism in exon 24 of the ABCA1 gene is represented by the nucleotide sequence SEQ ID NO: 111.

Mutation in exon 27

The mutation s-44e27 consists of the substitution of a guanine (G) with an adenine (A) at position 44 of exon 27, i.e., at position 604 with respect to the sequence SEQ ID NO: 14. The exon 27 carrying this polymorphism has the nucleotide sequence SEQ ID NO: 64. The cDNA carrying the polymorphism in exon 27 of the ABCA1 gene is represented by the nucleotide sequence SEQ ID NO: 112.

Mutations in exon 30

The mutation s-7e30 consists of the substitution of a guanine (G) with an adenine (A) at position 7 of exon 30, i.e., at position 6854 with respect to the sequence SEQ ID NO: 14. The exon 30 carrying this polymorphism has the nucleotide sequence SEQ ID NO: 65. The cDNA carrying the polymorphism in exon 30 of the ABCA1 gene is represented by the nucleotide sequence SEQ ID NO: 113.

The mutation s-166e30 consists of the substitution of a guanine (G) with a thymine (T) at position 166 of exon 30, i.e., at position 7013 with respect to the sequence SEQ ID NO: 14. The exon 30 carrying this polymorphism has the nucleotide sequence SEQ ID NO: 66. The cDNA corresponding to the mutation in exon 30 of the ABCA1 gene is represented by the nucleotide sequence SEQ ID NO: 114.

Mutation in intron 31

The mutation s-30i31 consists of the substitution of a thymine (G) with a guanine (G) at position 30 of the 5′ end of intron 31, i.e., at position 1307 with respect to the sequence SEQ ID NO: 15. The nucleotide sequence of the intron 31 carrying this polymorphism is represented in the sequence SEQ ID NO: 67.

Mutations in intron 32

The mutation s-m142i32 consists of the substitution of a cytosine (C) with a thymine (T) at position −142 upstream of the 3′ end of intron 32, i.e., at position 3600 with respect to the sequence SEQ ID NO: 15. The nucleotide sequence of the intron 32 carrying this polymorphism is represented in the sequence SEQ ID NO: 68.

The mutation s-m130i32 consists of an insertion of two nucleotides at position −130 upstream of the 3′ end of intron 32, i.e., at position 3611 with respect to the sequence SEQ ID NO: 15. The nucleotide sequence of the intron 32 carrying this polymorphism is represented in the nucleotide sequence SEQ ID NO: 69.

The mutation s-m26i32 consists of the substitution of a guanine (G) with a cytosine (C) at position −26 upstream of the 3′ end of intron 32, i.e., at position 3716 with respect to the sequence SEQ ID NO: 15. The nucleotide sequence of the intron 32 carrying this polymorphism is represented in the nucleotide sequence SEQ ID NO: 70.

Mutation in intron 33

The mutation s-m74i33 consists of the substitution of a thymine (T) with an adenine (A) at position −74 upstream of the 3′ end of intron 33, i.e., at position 5247 with respect to the sequence SEQ ID NO: 15. The nucleotide sequence of the intron 33 carrying this polymorphism is represented in the sequence SEQ ID NO: 71.

Mutation in exon 34

The mutation s-62e34 consists of the substitution of a guanine (G) with an adenine (A) at position 62 of exon 34, i.e., at position 5382 with respect to the sequence SEQ ID NO: 15. The exon 34 carrying this polymorphism has the nucleotide sequence SEQ ID NO: 72. The cDNA carrying the polymorphism in exon 34 of the ABCA1 gene is represented by the nucleotide sequence SEQ ID NO: 115 and encodes a mutated ABCA1 protein of SEQ ID NO: 135 in which an arginine (R) has been replaced with a lysine (K) at position 1587.

The analysis of this polymorphism in a CCSMI (case-control study of myocardial infarction) population, which comprises a study of patients having survived a myocardial infarction and of control subjects of caucasian type (Parra et al., Arteriosclerosis and Thrombosis, (1992) 12(6), 701-707), shows that this polymorphism is associated significantly with an increased risk of myocardial infarction (F=18.6 p<0.0001).

This polymorphism appears, moreover, to be associated with lower mean levels of HDL cholesterol, and of apolipoprotein A-I and A-II. In particular, the presence of the allele A of the s-62e34 type is associated with a decrease of approximately 5 mg/dl in the apolipoprotein A-I level.

The polymorphism s-62e34 according to the invention, which causes the replacement of an arginine (R) with a lysine (K) at position 1587 of the ABCA1 protein, constitutes a functional mutation which affects the HDL cholesterol, apolipoprotein A-I and apolipoprotein A-II levels, and predisposes to a cardiovascular risk.

Mutations in exon 36

The mutation s-77e36 consists of the substitution of a guanine (G) with an adenine (A) at position 77 of exon 36, i.e., at position 1064 with respect to the sequence SEQ ID NO: 16. The exon 36 carrying this polymorphism has the nucleotide sequence SEQ ID NO: 73. The cDNA carrying the polymorphism in exon 36 of the ABCA1 gene is represented by the nucleotide sequence SEQ ID NO: 1 16 and encodes a mutated ABCA1 protein of SEQ ID NO: 136 in which a valine (V) is substituted with an isoleucine (I) at position 1674.

Mutation in intron 36

The mutation s-55i36 consists of the substitution of a thymine (T) with a cytosine (C) at position 55 of the 5′ end of intron 36, i.e., at position 1220 with respect to the sequence SEQ ID NO: 16. The intron 36 carrying this polymorphism has the partial nucleotide sequence SEQ ID NO: 74.

Mutation in exon 38

The mutation s-64e38 consists of the substitution of a thymine (T) to a cytosine (C) at position 64 of exon 38, i.e., at position 835 with respect to the sequence SEQ ID NO: 17. The exon 38 carrying this polymorphism has the nucleotide sequence SEQ ID NO: 75. The cDNA carrying the polymorphism in exon 38 of the ABCA1 gene is represented by the nucleotide sequence SEQ ID NO: 117.

Mutations in intron 39

The mutation s-251 i39 consists of the substitution of a thymine (T) with a guanine (G) at position 251 of the 5′ end of intron 39, i.e., at position 375 with respect to the sequence SEQ ID NO: 18. The partial nucleotide sequence of the intron 39 carrying this polymorphism is represented in the sequence SEQ ID NO: 76.

The mutation s-252i39 consists of the substitution of a thymine (T) with a cytosine (C) at position 252 of the 5′ end of intron 39, i.e., at position 376 with respect to the sequence SEQ ID NO: 18. The partial nucleotide sequence of the intron 39 carrying this polymorphism is represented in the sequence SEQ ID NO: 77.

Mutation in intron 40

The mutation s-m119i40 consists of the substitution of a cytosine (C) to a thymine (T) at position −119 upstream of the 3′ end of intron 40, i.e., at position 710 with respect to the sequence SEQ ID NO: 19. The nucleotide sequence of the intron 40 carrying this polymorphism is represented in the sequence SEQ ID NO: 78.

Mutation in intron 44

The mutation s-m69i44 consists of the substitution of a thymine (T) with a cytosine (C) at position −69 upstream of the 3′ end of intron 44, i.e., at position 108 with respect to the sequence SEQ ID NO: 20. The partial nucleotide sequence of the intron 44 carrying this polymorphism is represented in the nucleotide sequence SEQ ID NO: 79.

Mutation in exon 45

The mutations-114e45 consists of the substitution of a cytosine (C) with a thymine (T) at position 114 of exon 45, i.e., at position 290 with respect to the sequence SEQ ID NO: 20. The exon 45 carrying this polymorphism has the nucleotide sequence SEQ ID NO: 80. The cDNA carrying the polymorphism in exon 45 of the ABCA1 gene is represented by the nucleotide sequence SEQ ID NO: 118.

Mutation in intron45

The mutation s-m34i45 consists of the substitution of a cytosine (C) with a thymine (T) at position −34 upstream of the 3′ end of intron 45, i.e., at position 343 with respect to the sequence SEQ ID NO: 21. The partial nucleotide sequence of the intron 45 carrying this polymorphism is represented in the sequence SEQ ID NO: 81.

Mutations in intron 47

The mutation s-13i47 consists of the substitution of an adenine (A) with a guanine (G) at position 13 of the 5′ end of intron 47, Le., at position 1068 with respect to the sequence SEQ ID NO: 21. The partial nucleotide sequence of the intron 47 carrying this polymorphism is represented in the sequence SEQ ID NO: 82.

The mutation s-86i47 consists of the substitution of an adenine (A) with a cytosine (C) at position 86 of the 5′ end of intron 47, i.e., at position 1141 with respect to the sequence SEQ ID NO: 21. The partial nucleotide sequence of the intron 47 carrying this polymorphism is represented in the sequence SEQ ID NO: 83.

Mutations in exon 49

The mutation s-377e49 consists of the substitution of a thymine (T) with a cytosine (C) at position 377 of exon 49, i.e., at position 571 with respect to the sequence SEQ ID NO: 22. The exon 49 carrying this polymorphism has the nucleotide sequence SEQ ID NO: 84. The cDNA carrying the polymorphism in exon 49 of the ABCA1 gene is represented by the nucleotide sequence SEQ ID NO: 119.

The mutation s-833e49 consists of the substitution of a cytosine (C) with a thymine (T) at position 833 of exon 49, i.e., at position 1027 with respect to the sequence SEQ ID NO: 22. The exon 49 carrying this polymorphism has the nucleotide sequence SEQ ID NO: 85. The cDNA carrying the polymorphism in exon 49 of the ABCA1 gene is represented by the nucleotide sequence SEQ ID NO: 120.

The mutation s-1580e49 consists of the substitution of a cytosine (C) with a thymine (T) at position 1580 of exon 49, i.e., at position 1773 with respect to the sequence SEQ ID NO: 22. The exon 49 carrying this polymorphism has the nucleotide sequence SEQ ID NO: 86. The cDNA carrying the polymorphism in exon 49 of the ABCA1 gene is represented by the nucleotide sequence SEQ ID NO: 121.

The mutation s-1791e49 consists of the substitution of an adenine (A) with a thymine (T) at position 1791 of exon 49, i.e., at position 1985 with respect to the sequence SEQ ID NO: 22. The exon 49 carrying this polymorphism has the nucleotide sequence SEQ ID NO: 87. The cDNA carrying the polymorphism in exon 49 of the ABCA1 gene is represented by the nucleotide sequence SEQ ID NO: 122.

The mutation s-2034e49 consists of the substitution of an adenine (A) with a guanine (G) at position 2034 of exon 49, i.e., at position 2228 with respect to the sequence SEQ ID NO: 22. The exon 49 carrying this polymorphism has the nucleotide sequence SEQ ID NO: 88. The cDNA carrying the polymorphism in exon 49 of the ABCA1 gene is represented by the nucleotide sequence SEQ ID NO: 123.

The mutation s-2049e49 consists of the substitution of a cytosine (C) with a thymine (T) at position 2049 of exon 49, i.e., at position 2243 with respect to the sequence SEQ ID NO: 22. The exon 49 carrying this polymorphism has the nucleotide sequence SEQ ID NO: 89. The cDNA carrying the polymorphism in exon 49 of the ABCA1 gene is represented by the nucleotide sequence SEQ ID NO: 124.

The mutation s-2449e49 consists of the substitution of an adenine (A) with a guanine (G) at position 2449 of exon 49, i.e., at position 2643 with respect to the sequence SEQ ID NO: 22. The exon 49 carrying this polymorphism has the nucleotide sequence SEQ ID NO: 90. The cDNA carrying the polymorphism in exon 49 of the ABCA1 gene is represented by the nucleotide sequence SEQ ID NO: 125.

The mutation s-2843e49 consists of the substitution of a guanine (G) with an adenine (A) at position 2843 of exon 49, i.e., at position 3037 with respect to the sequence SEQ ID NO: 22. The exon 49 carrying this polymorphism has the nucleotide sequence SEQ ID NO: 91. The cDNA carrying the polymorphism in exon 49 of the ABCA1 gene is represented by the nucleotide sequence SEQ ID NO: 126.

Certain polymorphisms found in the coding regions, which correspond essentially to substitution s of a single nucleotide located on the third base of the codons of the open reading frame of ABCA1, do not cause modifications with regard to the nature of the amino acid encoded, given the rules of genetic degeneracy in humans, which are well known to a person skilled in the art. The polymorphisms which do not modify the amino acid sequence can, however, have an important biological function, which can have an effect on the concentration of circulating HDL and, therefore, of apolipoprotein A-I or A-II. Such polymorphisms can, for example, affect the regulation of transcription or translation, for example by acting on mRNA stability, splicing, the rate of transcription and translation, and the accuracy of translation.

OTHER POLYMORPHISMS

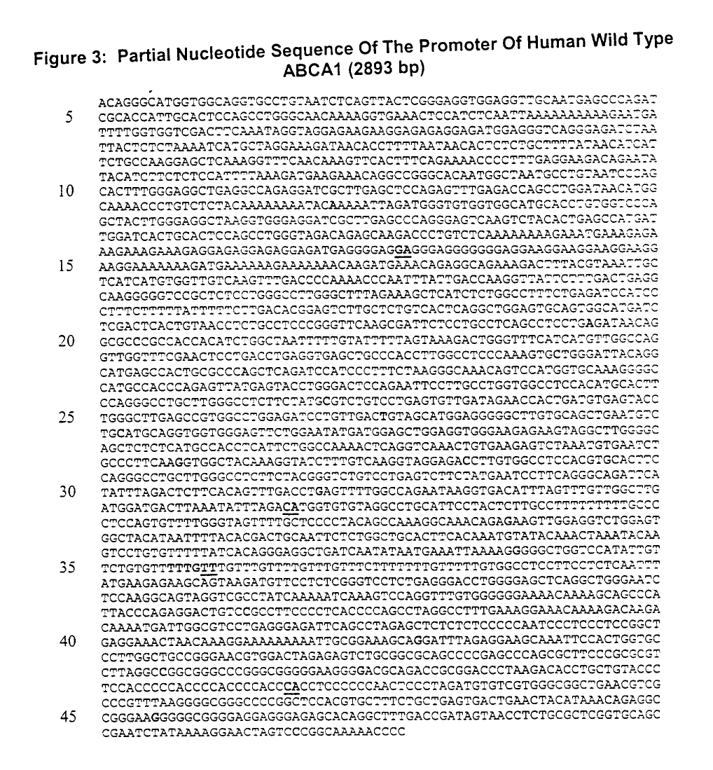

According to the invention, other polymorphisms have been discovered in the promoter region upstream of the human ABCA1 gene. The polymorphic positions are represented in FIG. 3 by nucleotides marked in bold (and underlined when they are bases framing an insertion) on the sequence of the promoter of the wild-type human ABCA1 gene of sequence SEQ ID NO: 23.

Table 3 presents the allelic polymorphisms in the promoter sequence of the human ABCA1 gene according to the present invention. The name, wild-type and mutant alleles, polymorphic positions and frequency of appearance of the allelic polymorphisms in two groups of population, the Caucasian population and the Japanese population, are indicated.

TABLE 3
Allelic Polymorphisms In The Promoter Of The Human ABCA1 Gene

| | | | | | | | Frequence In The | |
|---|---|---|---|---|---|---|---|---|
| | | | | | | | Population | |
| Name | Location | Position | 5′-Sequence | Allele 1 | Allele 2 | 3′-Seqence | Caucasian | Japanese |
| s-2389p | Promoter | −2389 | AAAAAAATAC | G | A | AAAATTAGAT | 29.60% | 33.30% |
| sins-2176-2177p | Promoter | −2176 | TGAGGGGAGG | GGAGGGGAGG | - | AGGGAGGGGG | 33.30% | 33.30% |
| s-1814p | Promoter | −1814 | CAGCCTCCTG | A | G | GATAACAGGC | 38.70% | 21.00% |
| s-1801p | Promoter | −1801 | TAACAGGCGC | C | T | CGCCACCACA | 41.90% | 66.10% |
| s-1652p | Promoter | −1652 | CACTGCGCCC | A | G | GCTCAGATCC | 18.70% | 21.00% |
| s-1506p | Promoter | −1506 | CTCTTCTATG | G | C | GTCTGTCCTG | 17.70% | 17.70% |
| s-1395p | Promoter | −1395 | TGAATGTCTG | C | T | ATGCAGGTGG | n.d. | 46.80% |
| s-1252p | Promoter | −1252 | TGCCCTTCAA | G | A | GTGGCTACAA | 27.30% | n.d. |
| s-1217p | Promoter | −1217 | AGGTAGGAGA | C | T | CTTGTGGCCT | 6.80% | 31.30% |
| s-1099p | Promoter | −1099 | AGTTTGACCT | G | T | AGTTTTGGCC | 9.10% | n.d. |
| s-1034-1035p | Promoter | −1034 | ATATTTAGAC | AT | - | ATGGTGTGTA | 25.00% | 19.40% |
| s-940p | Promoter | −940 | GGCAAACAGA | T | G | AAGTTGGAGG | 45.00% | 51.60% |
| s-803p | Promoter | −803 | AAATTAAAAG | G | A | GGGCTGGTCC | 14.60% | 51.90% |
| sΔ-777-774p | Promoter | −777 | GTTCTGTGTT | TTTG | - | TTTGTTTGTT | 11.50% | 5.00% |
| sΔ-773-765p | Promoter | −773 | TGTGTTTTTG | - | TTTGTTTGT | TTTGTTTGTT | 23.10% | 16.70% |
| s-564p | Promoter | −564 | GAGGACTGTC | C | T | GCCTTCCCCT | 21.40% | 32.80% |
| s-407p | Promoter | −407 | GCGGAAAGCA | G | C | GATTTAGAGG | 26.60% | 18.30% |
| s-302p | Promoter | −302 | CGTCTTAGGC | C | T | GGCGGGCCCG | 10.90% | 13.30% |
| s-278p | Promoter | −278 | GGGGGAAGGG | G | C | ACGCAGACCG | 34.40% | 33.30% |
| sΔ-223-219p | Promoter | −223 | CACCCCACCC | - | CACCC | ACCTCCCCCC | 10.90% | 13.30% |
| s-99p | Promoter | −99 | AGGCCGGGAA | G | C | GGGGCGGGGA | 41.70% | 35.00% |
| s-14p | Promoter | −14 | GGAACTAGTC | C | T | CGGCAAAAAC | 20.00% | 4.20% |

The polymorphism S-2389p consists of the substitution of a guanine (G) with an adenine (A) at position 505 of the first nucleotide of the sequence SEQ ID NO: 23, or at position −2389 with respect to the +1 transcription start site. The nucleic acid comprising the polymorphism S-2389p in the regulatory sequence of the ABCA1 gene is represented by the nucleotide sequence SEQ ID NO: 137.

The polymorphism Sins-2176-2177p consists of an insertion of the sequence GGAGGGGAGG at position 717 of the first nucleotide of the sequence SEQ ID NO: 23, or at position −2176 with respect to the transcription start site. The nucleic acid comprising the polymorphism Sins-2176-2177p in the regulatory sequence of the ABCA1 gene is represented by the nucleotide sequence SEQ ID NO: 138.

The polymorphism S-1814p consists of the substitution of an adenine (A) with a guanine (G) at position 1080 of the first nucleotide of the sequence SEQ ID NO: 23, or at position −1814 with respect to the +1 transcription start site. The nucleic acid comprising the polymorphism S-1 814p in the regulatory sequence of the ABCA1 gene is represented by the nucleotide sequence SEQ ID NO: 139.

The polymorphism S-1801 p consists of the substitution of a cytosine (C) with a thymine (T) at position 1093 of the first nucleotide of the sequence SEQ ID NO: 23, or at position −1801 with respect to the +1 transcription start site. The nucleic acid comprising the polymorphism S-1801 p in the regulatory sequence of the ABCA1 gene is represented by the nucleotide sequence SEQ ID NO: 140.

The polymorphism S-1652p consists of the substitution of an adenine (A) with a guanine (G) at position 1242 of the first nucleotide of the sequence SEQ ID NO: 23, or at position −1652 with respect to the +1 transcription start site. The nucleic acid comprising the polymorphism S-1652p in the regulatory sequence of the ABCA1 gene is represented by the nucleotide sequence SEQ ID NO: 141.

The polymorphism S-1506p consists of the substitution of a guanine (G) with a cytosine (C) at position 1388 of the first nucleotide of the sequence SEQ ID NO: 23, or at position −1506 with respect to the +1 transcription start site. The nucleic acid comprising the polymorphism S-1 506p in the regulatory sequence of the ABCA1 gene is represented by the nucleotide sequence SEQ ID NO: 142.

The polymorphism S-1395p consists of the substitution of a cytosine (C) with a thymine (T) at position 1499 of the first nucleotide of the sequence SEQ ID NO: 23, or at position −1395 with respect to the +1 transcription start site. The nucleic acid comprising the polymorphism S-1395p in the regulatory sequence of the ABCA1 gene is represented by the nucleotide sequence SEQ ID NO: 143.

The polymorphism S-1252p consists of the substitution of a guanine (G) with an adenine (A) at position 1642 of the first nucleotide of the sequence SEQ ID NO: 23, or at position −1252 with respect to the +1 transcription start site. The nucleic acid comprising the polymorphism S-1252p in the regulatory sequence of the ABCA1 gene is represented by the nucleotide sequence SEQ ID NO: 144.

The polymorphism S-1217p consists of the substitution of a cytosine (C) with a thymine (T) at position 1677 of the first nucleotide of the sequence SEQ ID NO: 23, or at position −1217 with respect to the +1 transcription start site. The nucleic acid comprising the polymorphism S-1217p in the regulatory sequence of the ABCA1 gene is represented by the nucleotide sequence SEQ ID NO: 145.

The polymorphism S-1099p consists of the substitution of a guanine (G) with a thymine (T) at position 1795 of the first nucleotide of the sequence SEQ ID NO: 23, or at position −1099 with respect to the +1 transcription start site. The nucleic acid comprising the polymorphism S-1099p in the regulatory sequence of the ABCA1 gene is represented by the nucleotide sequence SEQ ID NO: 146.

The polymorphism Sins-1034-1035p consists of an insertion of an adenine and of a thymine (AT) at position 1859 of the first nucleotide of the sequence SEQ ID NO: 23, or at position −1034 with respect to the +1 transcription start site. The nucleic acid comprising the polymorphism Sins-1034-1035p in the regulatory sequence of the ABCA1 gene is represented by the nucleotide sequence SEQ ID NO: 147.

The polymorphism S-940p consists of the substitution of a thymine (T) with a guanine (G) at position 1954 of the first nucleotide of the sequence SEQ ID NO: 23, or at position −940 with respect to the +1 transcription start site. The nucleic acid comprising the polymorphism S-940p in the regulatory sequence of the ABCA1 gene is represented by the nucleotide sequence SEQ ID NO: 148.

The polymorphism S-803p consists of the substitution of a guanine (G) with an adenine (A) at position 2091 of the first nucleotide of the sequence SEQ ID NO: 23, or at position −803 with respect to the +1 transcription start site. The nucleic acid comprising the polymorphism S-803p in the regulatory sequence of the ABCA1 gene is represented by the nucleotide sequence SEQ ID NO: 149.

The polymorphism S-777-774p consists of a deletion of a four nucleotide TTTG fragment at position 2117 of the first nucleotide of the sequence SEQ ID NO: 23, or at position -774 with respect to the +1 transcription start site. The nucleic acid comprising the polymorphism S-777-774p in the regulatory sequence of the ABCA1 gene is represented by the nucleotide sequence SEQ ID NO: 150.

The polymorphism S-773-765p consists of a deletion of a nine nucleotide TTTGTTTGT fragment at position 2121 of the first nucleotide of the sequence SEQ ID NO: 23, or at position -765 with respect to the +1 transcription start site. The nucleic acid comprising the polymorphism S-773-765p in the regulatory sequence of the ABCA1 gene is represented by the nucleotide sequence SEQ ID NO: 151.

The polymorphism S-564p consists of the substitution of a cytosine (C) with a thymine (T) at position 2330 of the first nucleotide of the sequence SEQ ID NO: 23, or at position -564 with respect to the +1 transcription start site. The nucleic acid comprising the S-564p polymorphism in the regulatory sequence of the ABCA1 gene is represented by the nucleotide sequence SEQ ID NO: 152.

The polymorphism S-407p consists of the substitution of a guanine (G) with a cytosine (C) at position 2487 of the first nucleotide of the sequence SEQ ID NO: 23, or at position -407 with respect to the +1 transcription start site. The nucleic acid comprising the S-407p polymorphism in the regulatory sequence of the ABCA1 gene is represented by the nucleotide sequence SEQ ID NO: 153.

The polymorphism S-302p consists of the substitution of a cytosine (C) with a thymine (T) at position 2592 of the first nucleotide of the sequence SEQ ID NO: 23, or at position -302 with respect to the +1 transcription start site. The nucleic acid comprising the S-302p polymorphism in the regulatory sequence of the ABCA1 gene is represented by the nucleotide sequence SEQ ID NO: 154.

The polymorphism S-278p consists of the substitution of a guanine (G) with a cytosine (C) at position 2616 of the first nucleotide of the sequence SEQ ID NO: 23, or at position -278 with respect to the +1 transcription start site. The nucleic acid comprising the S-278p polymorphism in the regulatory sequence of the ABCA1 gene is represented by the nucleotide sequence SEQ ID NO: 155.

The polymorphism S-223-219p consists of a deletion of a five nucleotide CACCC fragment at position 2671 of the first nucleotide of the sequence SEQ ID NO: 23, or at position -219 with respect to the +1 transcription start site. The nucleic acid comprising the polymorphism S-223-219p in the regulatory sequence of the ABCA1 gene is represented by the nucleotide sequence SEQ ID NO: 156.

The polymorphism S-99p consists of the substitution of a guanine (G) with a cytosine (C) at position 2795 of the first nucleotide of the sequence SEQ ID NO: 23, or at position -99 with respect to the +1 transcription start site. The nucleic acid comprising the S-99p polymorphism in the regulatory sequence of the ABCA1 gene is represented by the nucleotide sequence SEQ ID NO: 157.

The polymorphism S-14p consists of the substitution of a cytosine (C) with a thymine (T) at position 2880 of the first nucleotide of the sequence SEQ ID NO: 23, or at position -14 with respect to the +1 transcription start site. The nucleic acid comprising the S-14p polymorphism in the regulatory sequence of the ABCA1 gene is represented by the nucleotide sequence SEQ ID NO: 158.

According to a first aspect, the invention also relates to the nucleotide sequences of the ABCA1 gene comprising at least one biallelic polymorphism as described above.

Thus, a subject of the invention is a nucleic acid comprising a polynucleotide chosen from the group consisting of the nucleotide sequences SEQ ID NOS: 24-126 and 137-158 or a nucleic acid with a complementary sequence.

The invention also relates to a nucleic acid which hybridizes, under high stringency conditions, with a polynucleotide chosen from the group consisting of the nucleotide sequences SEQ ID NOS: 24-126 and 137-158, or to a nucleic acid with a complementary sequence.

The invention also relates to a nucleic acid having at least 9 consecutive nucleotides of a polynucleotide chosen from the group consisting of the nucleotide sequences SEQ ID NOS: 24-126 and 137-158, and comprising the polymorphic base, or a nucleic acid with a complementary sequence.

The invention also relates to a nucleic acid having at least 21 consecutive nucleotides of a polynucleotide chosen from the group consisting of the nucleotide sequences SEQ ID NOS: 24-126 and 137-158, and comprising the polymorphic base, or a nucleic acid with a complementary sequence.

A subject of the invention is also a nucleic acid having at least 21 consecutive nucleotides of a polynucleotide chosen from the group consisting of the nucleotide sequences SEQ ID NOS: 24-126 and 137-158, the polymorphic base being located at the center of the 21-base nucleotide fragment.

The invention also relates to a nucleic acid derived from a polynucleotide chosen from the group consisting of the nucleotide sequences SEQ ID NOS: 24-126 and 137-158 comprising the polymorphic sequence and at least two nucleotides located either side of the polymorphic position, or a nucleic acid with a complementary sequence.

Another subject of the invention relates to a nucleic acid encoding a polypeptide chosen from the group consisting of amino acid sequences SEQ ID NOS: 127-136.

The invention also relates to a polypeptide comprising an amino acid sequence chosen from the group consisting of the sequences SEQ ID NOS: 127-136.

A subject of the invention is also an antibody directed against a polymorphic ABCA1 polypeptide comprising an amino acid sequence chosen from the group consisting of the sequences SEQ ID NOS: 127-136, or a peptide fragment of the latter. The antibody according to the invention may comprise a detectable compound.

A subject of the invention is also a nucleic acid containing a polynucleotide encoding a polypeptide or a nucleic acid of interest functionally linked to a polynucleotide chosen from the group consisting of the nucleotide sequences SEQ ID NOS: 137-158.

A subject of the present invention is a recombinant cloning and/or expression vector which carries a nucleic acid comprising a polynucleotide chosen from the group consisting of the nucleotide sequences SEQ ID NOS: 24-126 and 137-158, and at least one of the polymorphisms as described above. The invention also relates to a host cell transformed with a nucleic acid comprising a polynucleotide chosen from the group consisting of the nucleotide sequences SEQ ID NOS: 24-126 and 137-158, and at least one of the polymorphisms as described above, or with the recombinant vector described above. The invention also encompasses a nonhuman transgenic mammal in which the somatic cells and/or germ cells have been transformed with a nucleic acid comprising a polynucleotide chosen from the group consisting of the nucleotide sequences SEQ ID NOS: 24-126 and 137-158, and at least one of the polymorphisms as described above, or with a recombinant vector as described above.

According to another aspect, a subject of the invention is a nucleotide probe or primer capable of hybridizing to nucleic acids of sequences SEQ ID NOS: 24-126 and 137-158 and comprising at least one polymorphic position, or to nucleic acids with a complementary sequence.

The nucleotide probes and primers comprise a polynucleotide of nucleotide sequence chosen from the group consisting of the sequences SEQ ID NOS: 24-126 and 137-158, or with a complementary sequence, containing one of the polymorphic bases according to the invention.

Generally, a nucleotide probe according to the invention has a length of 15, 16, 19 to 25, 35, 40, 50, 70, 80, 100, 200, 500, 1000, 1500 consecutive nucleotides of a polynucleotide of nucleotide sequence chosen from the group consisting of the sequences SEQ ID NOS: 24-126 and 137-158, and comprising at least one polymorphic base, or of a nucleic acid with a complementary sequence.

The nucleotide probes may consist of a nucleic acid comprising at least 21 consecutive nucleotides of a polynucleotide chosen from the group consisting of the nucleotide sequences SEQ ID NOS: 24-126 and 137-158. In one embodiment, the polymorphic base is located at the center of the 21-base nucleotide fragment.

In general a nucleotide primer according to the invention has a length of 8, 9, 10, 11, 12, 13, 14, 15, 16, 17, 18, 19, 20, 21, 22, 23, 24, 25 to 40 consecutive nucleotides of a polynucleotide of nucleotide sequence chosen from the group consisting of the sequences SEQ ID NOS: 24-126 and 137-158, and comprising at least one polymorphic base, or of a nucleic acid with a complementary sequence.

The base of the 3′ end of these primers may be complementary to a nucleotide located 2, 3, 4, 5, 6, 7, 8, 9, 10, 11, 12, 13, 14, 15, 16, 17, 18, 19, 20 nucleotides or more on the 3′ side from the position of the polymorphism according to the invention, present in one of the sequences SEQ ID NOS: 24-126 and 137-158 and/or of the complementary sequences thereof.

The definition of a nucleotide probe and primer according to the invention encompasses, therefore, oligonucleotides which hybridize, under the high stringency hybridization conditions defined above, with a nucleic acid chosen from the sequences SEQ ID NOS: 24-126 and 137-158, comprising at least one of the polymorphisms defined above, or with a sequence complementary to the latter.

Primers and of pairs of primers which make it possible to amplify various regions of the ABCA1 gene are, for example, the pair of primers represented by the primer of sequence SEQ ID NO: 159 (ACCGAAGTMGGAGTTGCTC) and the primer of sequence SEQ ID NO: 160 (ACTGTTTCACTAATACTTACTG).

A nucleotide primer or probe according to the invention can be prepared by any suitable method well known to a person skilled in the art, including by cloning and restriction enzyme action or, alternatively, by direct chemical synthesis according to techniques such as the phosphodiester method of Narang et al. (Methods Enzymol (1979) 68, 90-98) or of Brown et al. (Methods Enzymol (1979) 68, 109-151), the diethylphosphoramidite method of Beaucage et al. (Tetrahedron Lett. (1981) 22,1859-1862), or the solid support technique described in EU patent No. EP 0 707 592.

By way of illustration, suitable nucleotide primers according to the invention are, for example, primers in which the base at the 3 ′ end hybridizes with the base located immediately on the 3′ side of the polymorphic base of the fragment comprising the polymorphism. After a step of specific hybridization of the primer, an elongation step with a mixture of two dideoxynucleotides complementary to the polymorphic base of said polymorphism, for example labeled with two different fluorophores, then a step of detection of the fluorescence signal obtained, makes it possible to determine which of the two differently labeled fluorescent dideoxynucleotides has been incorporated, and to deduce the nature of the polymorphic base.

Each of the nucleic acids according to the invention, including the oligonucleotide probes and primers described above, can be labeled, if desired, by incorporating a label which can be detected by spectroscopic, photochemical, biochemical, immunochemical or chemical means.

For example, such labels can consist of radioactive isotopes (³²P, ³³P, ³H, ³⁵S), fluorescent molecules (5-bromodeoxyuridine, fluorescein, acetylamino-fluorene, digoxigenin) or ligands such as biotin.

The labeling of the probes may be carried out by incorporating labeled molecules into the polynucleotides by primer extension or by addition to the 5′ or 3′ ends.

Examples of nonradioactive labeling techniques for nucleic acid fragments are described in French patent No. FR 78 109 75 or in the articles by Urdea et al. (Nucl. Ac. Res. (1988) 11, 4937-4957) or Sanchez-pescador et al. (J. Clin. Microbiol. (1988) 26(10) 1934-1938).

Other approaches may be used for labeling and detecting the dideoxynucleotides. A homogenous phase method based on FRET (Fluorescence Resonance Energy Transfer) has been described, inter alia, by Chen and Kwok (Nucl Ac Res, 25 (1997) 347-353). According to this method, amplified genomic DNA fragments likely to contain polymorphisms are incubated with a primer labeled with fluoroscein at the 5′ end, in the presence of fluorescent dideoxynucleotide triphosphates and of a modified Taq polymerase. The labeled primer is extended by one base by incorporation of the labeled dideoxynucleotide specific for the allele present in the complementary genomic DNA sequence. At the end of this elongation reaction, the fluorescence intensities for the two label compounds of the labeled dideoxynucleotides are analyzed directly without separation or purification. The ratio for these two fluorescences makes it possible to determine the nucleotide used by the polymerase during the elongation, and to deduce the nature of the polymorphic base. This process can be carried out in a single tube, and the fluorescence modifications monitored in real time. Alternatively, the extension primer can be analyzed by MALDI-TOF mass spectrometry. The base located at the polymorphic position is identified by measuring the mass added to the microsequencing primer (Haff et al., Gen Res, 7 (1997) 378-388).

In one embodiment, probes according to the invention enable an amplification of the signal, like the probes described by Urdea et al. (Nucl. Acid. SympSer. (1991) 24, 197-200) or in European patent No EP-0,225,807.

The oligonucleotide probes according to the invention also may be used in Southern-type hybridizations to genomic DNA or Northern-type hybridizations to RNA.

The structural characteristics which make it possible to differentiate the normal sequences from the mutated sequences of ABCA1 (genomic sequences, messenger RNAs, cDNAs) are exploited in order to provide means for detecting the mutated sequences of ABCA1 in a sample, in particular in the form of probes which hybridize specifically with the mutated sequences of ABCA1, or pairs of primers which make it possible to selectively amplify the regions of the ABCA1 gene which carry the polymorphisms described above. Detecting the presence of these mutations can be carried out by discrimination of the length of the nucleic acid fragments amplified, by hybridization of the amplified fragments using the specific probes described above or by direct sequencing of the amplified fragments.

Nucleotide primers according to the invention can, for example, be immobilized on a support. In addition, it is possible to immobilize on a support, for example, in an ordered manner, multiple specific primers such as described above, each of these primers being suitable for detecting one of the polymorphisms of the ABCA1 gene according to the invention.

According to another aspect of the present invention, the polymorphisms of the ABCA1 gene are useful as genetic markers in studies of the association between the presence of a given allele in an individual and the predisposition of this individual to a given pathology and/or to a given response to a therapeutic molecule, for example, in the context of pharmacogenetic studies.

One subject of the present invention is therefore genetic markers comprising all or part of a nucleic acid chosen from the group consisting of the nucleotide sequences SEQ ID NOS: 24-126 and 137-158 or a nucleic acid with a complementary sequence, and comprising one of the polymorphisms according to the invention.

The genetic markers of the invention may be used for studies of the association of a given allele of the polymorphisms according to the invention with plasma HDL cholesterol, apolipoprotein A-I and apolipoprotein A-II levels in patients.

In one embodiment of the invention, the genetic markers are used for studies of association of the polymorphisms s-35e1a, s-16e1 b, s-76e1b, s-53e5, s-108e6, s-113e6, s-123e8, s-153e8, s-126e12, s-148e14, s-196e15, s-136e16, s-27e17, s10e7, s-4e18, s-40e19, s-123e22, s-54e23, s-98e24, s-149e24, s-44e27, s-7e30, s-166e30, s-62e34, s-77e36, s-64e38, s-114e45, s-377e49, s-833e49, s-1580e49, s-1791 e49, s-2034e49, s-2049e49, s-2449e49, and s-2843e49 plasma HDL cholesterol, apolipoprotein A-I and apolipoprotein A-l1 levels in patients.

In another embodiment of the invention, the genetic markers are used for studies of association of the polymorphisms s-113e6, s-1 96e15, s-136e16, s27e17, s-107e17, s-4e18, s-123e22, s-54e23, s-62e34, and s-77e36 with plasma HDL cholesterol, apolipoprotein A-I and apolipoprotein A-II levels in patients.

In one aspect, the present invention relates to genetic markers comprising all or part of a nucleic acid chosen from the group consisting of the nucleotide sequences SEQ ID NOS: 24-126 and 137-158 or a nucleic acid with a complementary sequence, for studies of the association of the polymorphisms as described above with a cardiovascular risk in patients, and more particularly a risk of myocardial infarction.

There are various types of method for the genetic analysis of complex character (phenotypes) (Lander et al. Science, 265 (1994) 2037-2048). In general, the biallelic polymorphisms according to the invention are useful in any one of the methods described in the prior art intended to demonstrate a statistically significant correlation between a genotype and a phenotype. For example, the biallelic polymorphisms can be used in linkage analyses and in allele sharing methods. Additionally, the biallelic polymorphisms according to the invention may be used for identifying genes associated with characteristics (phenotypes) which can be detected using association studies, this being an approach which does not require using families affected by the characteristic, and which also enables the identification of genes associated with complex and sporadic characteristics.

Other statistical methods using the biallelic polymorphisms according to the invention are, for example, those described by Forsell et al. (Biol Psychiatry 42 (1997) 898-903) Xiong et al. (Ann J Hum Genet 64 (1999) 629-640), Horvath et al. (Am J Hum Genet 63 (1998) 1886-1897), Sham et al. (Ann Hum Genet 59 (1995) 323-336) or Nickerson et al. (Genomics, 12 (1992) 377-387). In one aspect, the biallelic polymorphisms are used to identify the genes associated with a phenotype.

According to another aspect, a subject of the present invention is a method for detecting one of the polymorphisms described above in the nucleotide sequence of the ABCA1 gene of an individual.

The method according to the invention consists in determining the nucleotide sequence of all or part of the ABCA1 gene in said individual at one of the polymorphic positions of the invention and identifying the presence or absence of the SNP (single nucleotide polymorphism) at the position analyzed.

In one embodiment, the detection method according to the invention consists of determining the presence of a G or of an A at polymorphic position 113 of exon 6, the presence of a G or of an A at polymorphic position 196 of exon 15, the presence of a G or of an A at polymorphic position 136 of exon 16, the presence of an A or of a G at polymorphic position 107 of exon 17, the presence of a T or of a C at polymorphic position 27 of exon 17, the presence of a G or of a T at polymorphic position 4 of exon 18, the presence of a C or of a T at polymorphic position 123 of exon 22, the presence of a G or of a C at polymorphic position 54 of exon 23, the presence of a C or of a T at polymorphic position 62 of exon 34, the presence of a G or of an A at polymorphic position 77 of exon 36.

The detection method according to the present invention may also consists of determining the nucleotide sequence of the ABCA1 gene at position 62 of exon 34 and the SNP corresponding to the presence of a C or of a T.

According to a first embodiment of the method of the invention, the presence of at least one of the SNPs described above is identified by sequencing all or part of the ABCA1 gene of the individual at the polymorphic positions.

Amplification and sequencing primers can be constructed so as to hybridize with a given region of a sequence chosen from the group of sequence consisting of the sequences SEQ ID NOS: 24-126 and SEQ ID NOS: 137-158, or with a complementary sequence, and comprising at least one polymorphic position. Such sequencing primers are generally constructed to amplify fragments of approximately 250 to approximately 500 nucleotides, comprising at least one of the polymorphic positions according to the invention.

The fragments amplified, for example, by the PCR method, are then sequenced, and the sequence obtained is compared with the reference sequences SEQ ID NOS: 24-91 and 137 to 158 in order to determine whether or not one or more nucleotide deletions, additions or substitutions are found in the sequence amplified from the DNA contained in the biological sample originating from the individual tested.

The method of the invention allows the amplification of a nucleic acid containing at least one of the polymorphisms of the invention, and comprising the following steps:

a) bringing a sample, in which the presence of the ABCA1 nucleic acid is suspected, into contact with a pair of nucleotide primers for which the hybridization position is located, respectively, on the 5′ side on one strand, and on the 3′ side on the complementary strand, of the region of the target nucleic acid the amplification of which is desired, in the presence of the reagents required for the amplification reaction;

b) amplifying the region of the target nucleic acid; and

c) detecting the nucleic acid(s) amplified.

In order to implement the amplification method as defined above, use will advantageously be made of any one of the nucleotide primers defined according to the criteria mentioned above.

According to this embodiment, the invention relates, therefore, to a method for detecting one of the polymorphisms according to the invention in the ABCA1 gene of an individual, comprising the following steps:

a) sequencing, from a biological material originating from the individual to be tested, a nucleic acid fragment using at least one nucleotide primer which hybridizes with the sequence chosen from the sequences SEQ ID NOS: 24-91 or the sequences SEQ ID NOS: 137-158 according to the invention;

b) aligning the sequence obtained in a) with the sequence corresponding to the amplified sequence chosen from the sequences SEQ ID NOS: 24-91 or the sequences SEQ ID NOS: 137-158;

c) determining the presence of a polymorphism in the sequence of the nucleic acid fragment with respect to the reference sequences SEQ ID NOS: 24-91 or the reference sequences SEQ ID NOS: 137-158.

The method for detecting the presence of a polymorphism of the ABCA1 gene in an individual also comprises the steps of:

a) sequencing, from a biological material originating from the individual to be tested, all or part of the ABCA1 gene using a nucleotide primer which hybridizes with the sequence chosen from the [lacuna] SEQ ID NOS: 24-91 or the sequences SEQ ID NOS: 137-158;

b) aligning the nucleotide sequence obtained in a) with a sequence chosen from the group consisting of the sequences SEQ ID NOS: 1 and 3-23;

c) determining the nucleotide differences between the sequenced polynucleotide originating from the biological material of the individual to be tested and the reference sequences SEQ ID NOS: 1 and 3-23.

Another embodiment of the method according to the present invention uses the oligonucleotide probes as described above, which are capable of hybridizing specifically with a corresponding region of one of the sequences SEQ ID NOS: 24-91 or with a corresponding region of one of the sequences SEQ ID NOS: 137-158, and comprising at least one of the polymorphic bases according to the invention as described above, for the purpose of detecting the presence of one or more of the polymorphisms according to the invention in a sample originating from an individual.

In one embodiment, the nucleotide probes consist of a nucleic acid comprising at least 21 consecutive nucleotides of a polynucleotide chosen from the group consisting of the nucleotide sequences SEQ ID NOS: 24-126 and 137-158, the polymorphic base being located in the center of the 21-base nucleotide fragment.

The nucleotide probes according to this embodiment of the invention may be immobilized on a solid support. Such solid supports are well known to a person skilled in the art and comprise surfaces of microtitration plate wells covered with polystyrene beds, with magnetic beds, with nitrocellulose strips or with microparticles such as latex particles.

Consequently, the present invention also relates to a method for detecting the presence of a nucleic acid as described above in a sample, said method comprising the steps of:

a) bringing one or more nucleotide probes according to the invention into contact with the sample to be tested;

b) allowing one or more nucleotide probes to hybridize with complementary sequences present in the sample; and

c) detecting the hybrid(s) possibly formed between the probe(s) and the nucleic acid present in the sample.

The oligonucleotide probe(s) may be immobilized on a support and, may comprise a detectable label.

According to another aspect of the present invention, the methods for detecting the polymorphisms described above are used for diagnosing, in an individual, after an association study, a predisposition to develop a certain pathology, or for predicting, after a pharmacogenetic study, the pharmacological response of a patient to a given molecule.

Such the methods make it possible to diagnose a pathological risk linked to a deficiency of the ABCA1 transporter, or a nil or undesirable response to a given therapeutic molecule, in an individual who has been identified as carrying a given allele of at least one of the polymorphisms according to the invention.

The methods according to the invention are, for example, useful for diagnosing the predisposition of an individual to exhibit a coronary or cardiovascular risk, such as a risk of myocardial infarction or of atherosclerosis.

The methods according to the invention make it possible to determine whether or not an individual is at risk for developing a pathology linked to a deficiency in cholesterol metabolism, in particular, in the reverse transport of cholesterol, a risk of developing a familial HDL deficiency such as Tangier disease, or, more generally, a cardiovascular disease or a coronary disorder, such as a risk of myocardial infarction or of atherosclerosis.

Such methods comprise detecting, in cells from a biological sample originating from the individual to be tested, the presence or absence of a given allele of one of the polymorphisms according to the invention as described in Table 2, in the sequence of the human ABCA1 gene of sequence SEQ ID NOS: 3-22.

The methods according to the invention also make it possible to detect, in cells from a biological sample originating from the individual to be tested, the presence or absence of a given allele of one of the polymorphisms according to the invention as described in Table 3, in the regulatory sequence SEQ ID NO: 23 upstream of the ABCA1 gene, characterized by a modification in the expression of a gene whose expression is regulated by the ABCA1 promoter.

A subject of the present invention is a method for diagnosing a pathology linked to a deficiency of the ABCA1 transporter, which consists in detecting, in an individual, the presence of a given allele of one of the polymorphisms of the invention according to one of the detection methods described above.

The methods of the invention make it possible to diagnose the predisposition of an individual to develop a cardiovascular disease such as a myocardial infarction.

An additional subject of the present invention is a method and a kit for diagnosing a risk of myocardial infarction or a risk of cardiovascular disorder in an individual, consisting in detecting, in said individual, the presence of a nucleic acid comprising a polynucleotide of sequence SEQ ID NO: 72 or 115, and comprising a polymorphic base A at position 62 of exon 34, i.e., at position 5382 with respect to the sequence SEQ ID NO: 15.

A subject of the present invention is a method for predicting a pharmacological response linked to a variation of the ABCA1 transporter or of its regulation, which consists in detecting, in an individual, the presence of a given allele of at least one of the polymorphisms of the invention according to one of the detection methods described above.

By way of illustration, such SNP polymorphisms can be detected in order to determine the existence of a substitution of a nucleotide in the sequence of a nucleic acid corresponding to the ABCA1 gene of sequence SEQ ID NOS: 3-22 and/or in the regulatory sequence upstream of the ABCA1 gene of sequence SEQ ID NO: 23, or of the addition of one or more nucleotides or of the deletion of one or more nucleotides, in said sequences SEQ ID NOS: 3-22 or SEQ ID NO: 23.

A subject of the present invention is also the use of the oligonucleotide probes which hybridize specifically with a corresponding region of one of the sequences SEQ ID NOS: 24-126 or with a corresponding region of one of the sequences SEQ ID NOS: 137-158, comprising at least one of the polymorphic bases according to the invention as described above, for the purpose of diagnosing, in an individual, a predisposition to the development of a pathology linked to a deficiency in the reverse transport of cholesterol, such as myocardial infarction, atherosclerosis or Tangier disease, or more generally a cardiovascular disorder, or of predicting the pharmacological response of an individual to a given therapeutic molecule.

The diagnostic methods according to the present invention can advantageously be used in order to evaluate the efficacy of therapeutic products in the treatment of disorders linked to a deficiency of the ABCA1 transporter.

Individuals who are carriers of an allelic polymorphism of the ABCA1 gene can have, for example, differences in concentration of the various lipid variables which are commonly measured, such as the levels of HDL cholesterol, of triglycerides, of apolipoproteins A-I and A-II or of A-I and A-II lipoprotein particles, or differences in the regulation of the biosynthesis of the protein. The differences observed due to the presence of an allelic variant in an individual can be modified when a particular therapeutic agent is administered to said individual. The methods according to the present invention can, therefore, be useful for determining the clinical effect of a therapeutic substance and determining the therapeutic doses to be administered.

According to a final aspect, a subject of the present invention is a kit or pack for detecting the presence of one of the polymorphisms as described above, in the ABCA1 gene in an individual, said pack comprising:

a) one or more primers which hybridize with a region chosen from the sequences SEQ ID NOS: 24-126 or the sequences SEQ ID NOS: 137-158;

b) where appropriate, the means required for carrying out an amplification reaction for at least one polymorphic position of the present invention.

A subject of the invention is also a kit or pack for detecting one of the polymorphisms of the present invention in the ABCA1 gene, in an individual, comprising:

a) one or more oligonucleotide probes as defined above;

b) where appropriate, the reagents required for carrying out a hybridization reaction.

The nucleic acid fragments derived from any one of the nucleotide sequences SEQ ID NOS: 24-126 comprising one of the polymorphisms of the invention are, therefore, useful for detecting the presence of at least one polymorphic allele of the ABCA1 gene in a sample.

The probes according to the invention can also be used for detecting PCR amplification products or for detecting mismatches.

The detection pack or kit may comprise one or more probes that are immobilized on a support.

According to a second embodiment, the detection pack or kit is characterized in that the oligonucleotide probes comprise a detectable label.

According to one particular embodiment of the detection kit described above, such a kit will comprise a plurality of oligonucleotide probes in accordance with the invention, which may be used for detecting target sequences of interest or, alternatively, detecting mutations in the coding regions or in the noncoding regions of the nucleic acids according to the invention, more particularly of the nucleic acids of sequences SEQ ID NOS: 1 and 3-23 or the nucleic acids with a complementary sequence.

The probes according to the invention which are immobilized on a support can be organized in matrices such as “DNA chips”. Such ordered matrices have in particular been described in U.S. Pat. No. 5 143 854, and in applications WO 90/15 070 and WO 92/10 092.

Support matrices on which oligonucleotide probes have been immobilized at a high density are, for example, described in U.S. Pat. No. 5,412,087 and in PCT application No. WO 95/11 995.

The nucleotide primers according to the invention can be used for amplifying any one of the nucleic acids according to the invention, and more particularly all or part of a nucleic acid of sequences SEQ ID NOS: 24-126 and 137-158.

A subject of the invention is also a pack or kit for amplifying a nucleic acid according to the invention, and more particularly all or a part of a nucleic acid of sequences SEQ ID NOS: 1 and 3-23, said pack or kit comprising:

a) a pair of nucleotide primers in accordance with the invention, for which the hybridization position is located, respectively, on the 5′ side and on the 3′ side of the target nucleic acid the amplification of which is desired;

b) where appropriate, the reagents required for the amplification reaction.

Such an amplification pack or kit will advantageously comprise at least one pair of nucleotide primers as described above.

The invention is, in addition, illustrated, without however being limited, by the figures and examples below.

FIG. 1 illustrates the sequence SEQ ID NO: 1 corresponding to the nucleotide sequence of the transcript of the human ABCA1 gene;

FIG. 2 illustrates the amino acid sequence SEQ ID NO: 2 corresponding to the peptide sequence of the ABCA1 transporter protein;

FIG. 3 illustrates the regulatory sequence SEQ ID NO: 23 upstream of the human ABCA1 gene; the position of the various polymorphisms is indicated in bold.

### EXAMPLE 1: IDENTIFICATION OF THE POLYMORPHISMS OF ABCA1

The detection of polymorphisms in the sequences of the transcripts, or in the genomic sequences, of the ABCA1 gene can be carried out according to various protocols. One method is direct sequencing.

In the case of a transcript for which the structure of the corresponding gene is unknown or partially known, it is necessary to determine precisely its intron-exon structure and the genomic sequence of the corresponding gene. It involves, therefore, first isolating the ABCA1 clone(s) of genomic DNA corresponding to the transcript studied, sequencing the insert of the corresponding clone(s) and determining the intron-exon structure by comparing the sequence of the cDNA with that of the genomic DNA obtained. The technique for determining polymorphisms by direct sequencing consists in comparing the genomic sequences of the ABCA1 gene which have been obtained from at least 32 individuals from each population tested, Caucasian, African and Japanese. The divergences in sequence constitute polymorphisms. All those which modify the amino acid sequence of the wild-type protein are mutations likely to affect the function of said protein, which it is interesting to consider more particularly in the case/control association studies described in example 2.

Primers for amplifying the DNA of individuals were designed based on the nonrepetitive sequences of the intronic DNA of the ABCA1 gene, so as to include an amplification of the intron/exon junctions and of the bases essential for the formation of the secondary structure during the RNA splicing step, in the amplified fragments.

The genomic DNA of the individuals was amplified with the aid of the primers described above, using Qiagen's Star Taq® kit (Qiagen, Valencia, Calif.) or the Supertaq®) kit (Ambion, Austin, Tex.), using the hybridization conditions and amplification cycle conditions recommended by the manufacturer.

The amplified PCR products were purified using a kit sold by the company Qiagen, then sequenced by the Big Dye Terminator method on an ABI 3700 sequencer (Apllied Biosystems, Foster City, Calif.).

EXAMPLE 2: ANALYSIS OF THE POLYMORPHISMS OF THE ABCA1 GENE

The Case-Control Study of Myocardial Infarction (CCSMI) is a study of patients who have suffered a myocardial infarction and of control individuals originating from Northern Ireland, from England and from France (all of the Caucasian type).

The study was carried out in order to identify a possible association of exon polymorphisms such as for example s-16e1b, s-113e6, s-135e8, s-148e14, s-136e16, s-101e17, s-54e23, s-7e30, s-62e34, s-833e49 and s-2034e49, with plasma HDL cholesterol, ApoA-I and ApoA-II levels in controls and patients.

The genomic DNA of the individuals was amplified with the aid of the primers described above which make it possible to amplify specifically the exon of interest and the corresponding intron-exon junctions, using, for example, Qiagen's Star Taq® kit (Qiagen, Valencia, Calif.) or the Supertaq®) kit (Ambion, Austin, Tex.), under the hybridization conditions and amplification cycle conditions recommended by the manufacturer. Minisequencing reactions were carried out on these amplicons for each of the polymorphisms selected. The genotype of each of the controls and of the patients was thus determined for each of the polymorphisms.

These data were analyzed using the SAS software. The Hardy-Weinberg equilibrium was tested using a ² test with 1 df. The genotypic and allelic frequencies were compared between the groups using ² tests. The various phenotypic parameters were compared between the genotypes by ANOVA variance analysis.

We claim:

1. A nucleic acid comprising a polynucleotide chosen from the group consisting of SEQ ID NOS: 24-126 and 137-158 and nucleic acids complementary to SEQ ID NOS: 24-126 and 137-158.

2. A nucleic acid comprising at least 9 consecutive nucleotides of a polynucleotide chosen from the group consisting of SEQ ID NOS: 24-126 and nucleic acids complementary to SEQ ID NOS: 24-126, and comprising a polymorphism chosen from the group consisting of s-m39i3, s-53e5, s-113e6, s-135e8, s-24il3, s-148e14, s-81i14, s-115i14, s-196e15, s-136e16, s-18i19, s-m65i22, s-149e24, s-44e27, s-7e30, s-62e34, s-m74i34, s-77e36, s-2843e49, s-25i4, s-123e8, s-m13i9, s-126e12, s-m59i13, s-4e18, s-40e19, s-m10i19, s-123e22, s-149i23, s-m50i23, s-166e30, s-m142i32, s-m19i40, s-114e45 s-m34i45, s-833e49, s-1580e49, s-1791e49, s-2049e49, s-35e1a, s-108e6, s-m89i8 s-m104i9, s-m29i12, s-107e17, s-m68i17, s-98e24, s-30i31, s-251i39, s-13i47 s-2034e49, s-2449e49, s-76e1b, s-27e17, s-60i17, s-54e23, s-m26i32, s-55i36, s-64e38, s-252i39, s-m69i44, s-86i47, s-377e49, s-m41i9, s-16e1b, s-14i7, s-m61i9, and s-m130i32.

3. A nucleic acid comprising at least 9 consecutive nucleotides of a polynucleotide chosen from the group consisting of SEQ ID NOS: 137-158 and nucleic acids complementary to SEQ ID NOS: 137-158, and comprising a polymorphism in the promoter sequence of the ABCA1 gene SEQ ID NO: 23, said polymorphism selected from the group consisting of i) an A at positions 505, 1642, 2091, ii) a T at positions 1093,1499,1677, 1795, 2330, 2592, 2880, iii) a G at positions 1080, 1242, 1954, iv) a C at positions 1388, 2487, 2616, 2795, v) an insertion of GGAGGGGAGG at position 717, an AT insertion at position 1859, and vi) a deletion of TTTG at position 2117, a deletion of TTTGTTTGT at position 2121, and a deletion of CACCC at position 2671.

4. The nucleic acid according to claim 2, comprising at least 21 consecutive nucleotides of a polynucleotide chosen from the group consisting of SEQ ID NOS: 24-126 and nucleic acids complementary to SEQ ID NOS: 24-126, and comprising a polymorphism chosen from the group consisting of s-m39i3, s-53e5, s-113e6, s-135e8, s-24il3, s-148e14, s-81i14, s-115i14, s-196e15 s-136e16, s-18i19, s-m65i22, s-149e24, s-44e27, s-7e30, s-62e34, s-m74i34, s-77e36, s-2843e49, s-25i4, s-123e8, s-m13i9, s-126e12, s-m59i13, s-4e18, s-40e19, s-m10i19, s-123e22, s-149i23, s-m50i23, s-166e30, s-m142i32, s-m119i40, s-114e45, s-m34i45, s-833e49, s-1580e49, s-1791e49, s-2049e49, s-35e1a, s-108e6, s-m89i8, s-m104i9, s-m29i12, s-107e17, s-m68i17, s-98e24, s-30i31, s-251 i39, s-13i47, s-2034e49, s-2449e49, s-76e1 b, s-27e17, s-60i17, s-54e23, s-m26i32, s-55i36, s-64e38, s-252i39, s-m69i44, s-86i47, s-377e49, s-m41i9, s-16e1b, s-m14i7, s-m61i9, and s-m130i32.

5. The nucleic acid according to claim 3, comprising at least 21 consecutive nucleotides of a polynucleotide chosen from the group consisting of SEQ ID NOS: 137-158 and nucleic acids complementary to SEQ ID NOS: 137-158, and comprising a polymorphism in the promoter sequence of the ABCA1 gene SEQ ID NO: 23, said polymorphism selected from the group consisting of i) an A at positions 505,1642, 2091, ii) a T at positions 1093, 1499, 1677, 1795, 2330, 2592, 2880, iii) a G at positions 1080, 1242,1954, iv) a C at positions 1388, 2487, 2616, 2795, v) an insertion of GGAGGGGAGG at position 717, an AT insertion at position 1859, and vi) a deletion of TTTG at position 2117, a deletion of TTTGTTTGT at position 2121, and a deletion of CACCC at position 2671.

6. The nucleic acid according to claim 4, wherein the polymorphism is located at the center of said at least 21 consecutive nucleotides.

7. The nucleic acid according to claim 5, wherein the polymorphism is located at the center of said at least 21 consecutive nucleotides.

8. A nucleic acid, wherein said nucleic acid hybridizes under high stringency conditions with a polynucleotide selected from the group consisting of SEQ ID NOS: 24-126, SEQ ID NOS: 137-158, sequences complementary to SEQ ID NOS: 24-126, and sequences complementary to SEQ ID NOS: 137-158.

9. A nucleic acid, wherein said nucleic acid encodes a polypeptide selected from the group consisting of SEQ ID NOS: 127-136.

10. A nucleic acid comprising:
a) a first nucleic acid comprising a polynucleotide chosen from the group consisting of the nucleotide sequences SEQ ID NOS: 137-158; and
b) a second nucleic acid encoding a polypeptide or a nucleic acid of interest.

11. A polymorphic ABCA1 polypeptide, comprising a sequence selected from the group consisting of SEQ ID NOS: 127-136.

12. A nucleotide probe or primer, comprising a nucleic acid according to claim 2 or claim 3.

13. The nucleotide probe or primer according to claim 12, further comprising a detectable label.

14. The nucleotide probe or primer according to claim 12, wherein a nucleotide at the 3′ end of said probe or primer is complementary to a nucleotide located immediately on the 3′ side of the polymorphic base of one of the sequences selected from the group consisting of SEQ ID NOS: 24-126, SEQ ID NOS: 137-158, sequences complementary to SEQ ID NOS: 24-126, and sequences complementary to SEQ ID NOS: 137-158.

15. The nucleotide probe or primer according to claim 12, wherein a nucleotide at the 3′ end of said probe or primer is complementary to a nucleotide located between 2 and 20 nucleotides or more on the 3′ side of the polymorphic base of one of the sequences selected from the group consisting of SEQ ID NOS: 24-126, SEQ ID NOS: 137-158, sequences complementary to SEQ ID NOS: 24-126, and sequences complementary to SEQ ID NOS: 137-158.

16. The nucleotide probe or primer according to claim 12, wherein a nucleotide at the 3′ end of said probe or primer is complementary to a nucleotide located more than 20 nucleotides on the 3′ side of the polymorphic base of one of the sequences selected from the group consisting of SEQ ID NOS: 24-126, SEQ ID NOS: 137-158, sequences complementary to SEQ ID NOS: 24-126, and sequences complementary to SEQ ID NOS: 137-158.

17. The nucleotide probe or primer according to claim 12, wherein said probe or primer identifies a genetic marker.

18. A method for detecting a nucleic acid according to claim 1 in a sample, said method comprising:
a) contacting said sample with a pair of nucleotide primers for which the hybridization position is located, respectively, on the 5′ side and on the 3′ side of a target polymorphic position to be amplified, in the presence of reagents required for an amplification reaction;
b) performing the amplification reaction; and
c) detecting the nucleic acids amplified.

19. A method for detecting a nucleic acid according to claim 2 in a sample, said method comprising:
a) contacting said sample with a pair of nucleotide primers for which the hybridization position is located, respectively, on the 5′ side and on the 3′ side of a target polymorphic position to be amplified, in the presence of reagents required for an amplification reaction;
b) performing the amplification reaction; and
c) detecting the nucleic acids amplified.

20. The method according to claim 18, wherein the pair of nucleotide primers are chosen from the primers comprising a nucleic acid according to claim 2 or claim 3.

21. The method according to claim 19, wherein the pair of nucleotide primers are chosen from the primers comprising a nucleic acid according to claim 2 or claim 3.

22. A kit for performing the method according to claim 18, wherein said kit comprises a pair of nucleotide primers for which the hybridization position is located, respectively, on the 5′ side and on the 3′ side of a target polymorphic position to be amplified of the nucleic acid.

23. The kit according to claim 22, further comprising reagents required for an amplification reaction.

24. A kit for performing the method according to claim 19, wherein said kit comprises a pair of nucleotide primers for which the hybridization position is located, respectively, on the 5′ side and on the 3′ side of a target polymorphic position to be amplified of the nucleic acid.

25. The kit according to claim 24, further comprising reagents required for an amplification reaction.

26. The kit according to claim 22, wherein the pair of nucleotide primers are chosen from the primers comprising a nucleic acid according to claim 2 or claim 3.

27. The kit according to claim 24, wherein the pair of nucleotide primers are chosen from the primers comprising a nucleic acid according to claim 2 or claim 3.

28. A method for detecting the presence of a polymorphism of an ABCA1 gene in an individual, said method comprising:
a) sequencing, from a biological material originating from the individual to be tested, all or part of the ABCA1 gene using a nucleotide primer according to claim 12;
b) aligning the nucleotide sequence obtained in a) with a sequence chosen from the group consisting of SEQ ID NOS: 1 and 3-23; and
c) determining the nucleotide differences between the sequenced polynucleotide originating from the biological material of the individual to be tested and the reference sequences SEQ ID NOS: 1 and 3-23.

29. A kit for performing the method according to claim 28, said kit comprising means for sequencing all or part of the ABCA1 gene.

30. A method for detecting the presence of a polymorphism of a ABCA1 gene in an individual, said method comprising:
a) bringing one or more nucleic acid probes according to claim 12 into contact with a sample originating from the individual to be tested;
b) incubating said one or more nucleic acid probes with the sample under conditions that permit the probes to hybridize with complementary nucleic acids in the sample; and
c) detecting complexes formed between the probes and the complementary nucleic acid present in the sample.

31. The method according to claim 30, wherein said one or more probes are immobilized on a support.

32. A kit for detecting the presence of a nucleic acid comprising a polynucleotide chosen from the group consisting of SEQ ID NOS: 24-126 and 137-158 and nucleic acids complementary to SEQ ID NOS: 24-126 and 137-158., wherein said kit comprises at least one nucleotide primer or probe according to claim 12.

33. The kit according to claim 32, further comprising reagents for a hybridization reaction.

34. The kit according to claim 32, wherein said at least one nucleotide primer or probe is immobilized on a support.

35. A method for diagnosing a risk of cardiovascular disorder in an individual, comprising determining whether said individual has a nucleic acid comprising a polynucleotide of sequence SEQ ID NO: 72 or 115, wherein said polynucleotide comprises a polymorphic base A at position 5382 of SEQ ID NO: 15.

36. The method according to claim 35, wherein the cardiovascular disorder is a myocardial infarction.

37. A recombinant vector comprising the nucleic acid according to claim 1 or a portion thereof.

38. The recombinant vector according to claim 37, wherein said vector is an expression vector.

39. A host cell transformed with the nucleic acid according to claim 1 or a portion thereof.

40. A host cell transformed with the recombinant vector according to claim 37.

41. A nonhuman, transgenic animal, wherein somatic cells and/or germ cells of said animal have been transformed with the nucleic acid according to claim 1 or a portion thereof.

42. A nonhuman, transgenic animal, wherein somatic cells and/or germ cells of said animal have been transformed with the vector according to claim 37.

43. An antibody directed against the polymorphic ABCA1 polypeptide according to claim 11 or a fragment thereof.

44. The antibody according to claim 43, further comprising a detectable label.

45. A method for detecting the presence of a polymorphic ABCA1 protein in a sample, comprising:
a) contacting the sample an antibody according to claim 43 under conditions permitting antibody/antigen complexes to form; and
b) detecting the antigen/antibody complex formed.

46. A kit for performing the method according claim 45, said kit comprising:
a) an antibody directed against a polymorphic ABCA1 polypeptide, wherein said peptide comprises a sequence selected from the group consisting of SEQ ID NOS: 127-136; and
b) a reagent which enables the detection of the antigen/antibody.
